(19) United States
(12) Patent Application Publication
(10) Pub. No.: US 2003/0203373 A1
(43) Pub. Date: Oct. 30, 2003

(54) Method for identifying a pharmacologically active substance

(76) Inventors: Wolf-Dieter Schleuning, Berlin (DE); Torsten Schulz, Berlin (DE)

Correspondence Address: SUGHRUE MION, PLLC, 2100 PENNSYLVANIA AVENUE, N.W., WASHINGTON, DC, 20037, US

(21) Appl. No.: 10/201,288

(22) Filed: Jul. 24, 2002

(30) Foreign Application Priority Data
Feb. 20, 2002 (DE) 102 08 187.5

(51) Int. Cl. C12Q 1/68; G01N 33/53; G06F 19/00; G01N 33/48; G01N 33/50

(52) U.S. Cl. 435/6; 435/7.1; 702/20; 702/19

(57) ABSTRACT

Method for identifying a novel biologically active substance, which is based on defining the targeted property of the substance and selecting a reference organism, naturally displaying the targeted property.

The invention pertains to a method for identifying a commercially applicable, especially pharmacologically active substance.

The effect of a therapeutic is usually based on an active ingredient exhibiting a biological function in the target organism. This effect is mainly due to a carefully targeted site-specific influence on recognition structures within the body such as e.g. enzymes, channels, receptors or signal proteins and nucleic acids. Consequently, the development of new drugs can be alternatively based on the identification of new recognition structures (drug targets) or on providing already known active substances with optimized characteristics. Often these approaches are combined.

Developing new drugs traditionally is based on substances or substance compositions found in nature or on merely randomly synthesized substances, which are screened for a potential biological or chemical activity in cell culture or animal trials. Subsequently, substances being identified as promising candidates (the so-called lead substances) can be gradually modified while measuring the alterations of biological or chemical activity caused by the respective modifications. This way of screening for novel drugs derived from natural substances—so-called bioprospecting—today remains to be an important fundament of drug research (Cragg G M, Newman D J, Yang S S. Nature. Apr. 9, 1998;392(6676):535-7, 539-40: Bioprospecting for drugs; Balick M J. Ciba Found Symp 1994;185:4-18; discussion 18-24: Ethnobotany, drug development and biodiversity conservation—exploring the linkages).

Due to automatisation (so-called High Throughput Screening, HTS) and increasing knowledge about the site-specific modification of the revealed substances, the efficacy of this traditional “blind search” for active substances has been improved (Rosell S. Lakartidningen Dec. 17, 1997; 94(51-52):4938-41: An entire rain forest can be screened at pharmaceutical industry's laboratories). However, this approach still remains unsatisfactory in cost-profit relations: one successfully developed new drug is opposed by a hardly acceptable vast number of investigated but finally rejected substances (Grabley S., Thiericke R.: “Bioactive agents from natural sources: trends in discovery and application”, Adv Biochem Eng Biotechnol 1999; 64:101-54; Landro J A et al.: “HTS in the new millenium: the role of pharmacology and flexibility”, J Pharmacol Toxicol Methods July-August 2000; 44(1):273-89). Furthermore, it often turns out lateron, that a substance displays a special effect for an indication, that first was not observed during the initial synthesis and investigation.

The possibilities of combinatorial chemistry improve this approach of drug discovery. However, also this improvement is limited. Combinatorial chemistry is based on a systematically varied combination of modules which form a high number of related compounds—up to several millions—and at least partly—and to different degrees—are expected to display the desired activity (Gayo L M: “Solution-phase library generation: methods and applications in drug discovery”, Biotechnol Bioeng 1998 Spring;61(2):95-106; Bradley E K et al.: “A rapid computational method for lead evolution: description and application to alpha(1)-adrenergic antagonists” J Med Chem Jul. 13, 2000;43(14):2770-4).

The methodical search for novel drug substances (the so-called rational drug development) differs from the approach of “blind search”. It depends on the prior knowledge about the molecular mechanisms of the disease to be treated and a specific design of a substance interacting with the respective drug target on a molecular basis. Therewith the rational drug development especially combines bio- and chemo-informatical methods for the purpose of a methodical search for suitable drug candidates (Bajorath J.: “Rational drug discovery revisted: interfacing experimental programs with bio- and chemo-informatics”, Drug Discov Today Oct. 1, 2001;6 (19):989-995). Thus, this method first requires the identification of the structures being involved in pathogenesis—mostly proteins or their genetical grounds. Once the relevant protein-coding genes are identified, they can be inserted e.g. into vectors, microorganisms, plant- or animal-models in order to investigate the structure-function relationship of the proteins within these models. In order to characterize especially the relevant binding site(s) structural analyses using X-ray crystallography may be employed additionally.

The data about the properties of the target enable the subsequent selective search for and optimization of a well-fitting ligand as a future drug substance. This is possible by so-called structure-based design, which derives a lead structure of the ligand best fitting to the target, by employing computer-based three-dimensional models of the target (or its binding site(s)) as well as information about known molecules and structural elements or by using affinity selection methods such as DNA-libraries coding for peptides.

This approach of rational drug development is time- and cost-consuming especially due to the obligatory prior knowledge of the molecular mechanisms responsible for pathogenesis.

The decoding of the human genome as well as the enormous increase of knowledge about the genome of standard model systems in combination with meanwhile mostly fully automatized screening methods has led to new hopes for a more efficient and less time-consuming drug development. This drug development is increasingly based on the methodical approach of comparative genome analysis (comparative genomics).

The possible application of comparative genome analysis within drug development requires the prior identification of a pathogenesis-related gene, e.g. in the human. Subsequently, an orthologous animal gene of a model organism (e.g. the mouse) is identified by homology search in a gene bank. Site-directed manipulation and modification of the animal model then may allow for conclusions about the molecular mechanisms of pathogenesis in the human. Here, especially knock-out-models offer detailed information about the molecular properties of potential drug targets (Harris S., Foord S M.: “Transgenic gene knock-outs: functional genomics and therapeutic target selection.”, Pharmacogenomics November 2000;1(4):433-43).

Alternatively, the investigation can begin with the identification of a gene relevant for pathogenesis in an animal model, which is later introduced into a comparative genome analysis in order to identify an orthologous gene in humans. The respective gene product subsequently can be used as a target or can as such serve as a basis for further drug development.

This approach is disclosed in WO 00/45848, describing the use of the hedgehog-protein, which was previously characterized in model systems of developmental biology, for the treatment of bone- and cartilage-damages and neural defects in humans. Therefore, first the human gene product being orthologous to the animal hedgehog-protein was identified and thereafter the substance was optimized into a drug by conventional methods.

Although the possibilities of comparative genome analysis have led to new impulses in the previous years by facilitating the understanding of molecular backgrounds, initial hopes mostly turned out to be in vain. This is largely due to the fact, that even when possessing information about the molecular mechanisms of a disease, the directed development or identification of a suitable therapeutic substance remains to be difficult.

Thus, it is the problem of the invention to provide a method enabling an improved targeted identification of biologically active substances, which are active especially in humans.

This problem is solved by a method according to the independent claims. Advantageous further objectives of the inventions are subject of the dependent claims.

The basic idea of the method according to the invention is the search and identification of a reference organism exhibiting a special property and the subsequent genomic comparison with the target organism. The search for a suitable reference organism first requires the definition of desired physiological function, i.e. the biological or chemical effect. Secondly, the organism is identified, which displays the desired function in adaptation to its life in nature—i.e. in order to cope with physiological problems to solve in its natural environment. Consequently, the reference organism has developed physiological mechanisms to solve a specific problem, which also is to be solved by the active substance, which is to be found. Alternatively, also an already identified function can be used as a starting point.

In contrast to the known method of rational drug development the method according to the invention does not apply the comparative genome analysis for investigating the molecular mechanisms underlying the disease, but indirectly serves for the targeted search for novel structures within the body, which exhibit previously defined functions within the target organism.

A specific advantage of the inventive method is a remarkable reduction of the period of time necessary for the identification and development of a novel therapeutic substance, since a previous understanding of the molecular mechanisms of pathogenesis and the relevant structures is not obligatory. Additionally the revealed body-own active substances or therapeutically relevant structures exhibit a high specificity in most cases thereby reduce possible unwanted side-effects of a drug.

Thus, the first step of the method according to the invention is the selection of a reference organism, which—in adaptation to its natural habitats—possesses exactly those (physiological) characteristics, the desired substance shall have. As reference organism especially animal organisms, as well as for example plants or microorganisms are suitable. Also, specialized tissues of the target organisms as well as individual sub-populations may be summarized under the expression “reference organism”. They may e.g. express especially suitable allelic variants (single nucleotide polymorphisms, SNPs) of a desired feature.

Advantageously data bases in the fields of zoology, botany, microbiology, physiology, biochemistry, genetics or medicine can be used to identify suitable reference organisms. Examples of suitable data bases are “Biological Abstracts®”, “BIOSIS Previews®”, “CABCD”, “Current Contents Search®”, “Life Science Collection”, “Medline” and “Plant-Gene”. Of course, further sources such as specific literature, films, microfilms, acoustic and electronic data carriers are included in the range of suitable data sources.

Subsequent to the identification of at least one suitable reference organism, the genes responsible for expressing the desired characteristics are identified. First, gene expression pattern of tissues of interest can be investigated by using differential display or microarrays. This usually already leads to a reduction of the number of potentially interesting genes. Then, a precise idea of the gene expression pattern of the tissues and cells of the selected biological model can be generated by employing modern high throughput DNA-sequencing e.g. in combination with ESI-MS/mass spectroscopy of proteins separated by highly effective resolution methods. Starting from this expression library, a cDNA-library can be generated by using established methods.

Subsequently, a genomic comparison of this cDNA-library with the genetic information of the target organism being available from data bases can be conducted. Therefore, the one skilled in the art, may apply e.g. bioinformatical software programs that have been developed for comparative genomic analysis. Starting from the orthologous gene identified in the target organism the corresponding gene product can be identified. This identification is preferably enabled by comparisons of ESTs (Expressed Sequence Tags).

The gene product—e.g. from a human—subsequently might be used directly as a therapeutic. However, it may be advantageous to prior modify or modulate the identified genetic sequence e.g. in case the gene is present in the inactive state or to use the gene product for the development of a therapeutic substance.

The application of functional modifications and their impact on protein structure as well as the further down stream development can be preferably supported by employment of methods of structural analysis or molecular modeling.

Alternatively and additionally the data derived from the identified biologically active substance might be used for or lead to the identification of a lead structure interacting therewith.

In a preferred embodiment of the invention the identification of an orthologous substance or a target molecule (drug target) starts with the establishment of an EST-library of the relevant tissues of the selected reference organism. When the library is established these tissues preferably are in a physiological state, in which—due to the organism's adaptation to the actual living conditions—most probably a peptide or protein of a physiological function is expressed, that is—at least largely—similar to the desired biologically active substance in the target organism. Subsequently the relevant peptides or proteins can be identified. Then, the EST-libraries created therewith as well as the information derived from comparative genome analysis of the target organism can be used to identify orthologous structures, e.g. in a human. Examples of these structures are pharmacologically active substances, lead structures or target molecules (drug targets).

### EXAMPLES

I. Human BPP

The control of blood pressure in the human is mainly accomplished by the so-called renin-angiotensin-aldosteron system, that inter alia becomes activated in case of a blood pressure drop:

Due to the adaptation of circulation, adrenalin and noradrenalin quickly increase in healthy persons in case of physical stress. In patients with acute heart insufficiency the arterial blood pressure declines as a consequence of a strong diminution of the heart time volume. In a reflex of neurohumoral counteraction the secretion of noradrenalin from the adrenal body is stimulated by the baroreceptor reflex and by sympathetic nerve fibers. Additionally a small amount of noradrenalin, acting as a neuronal transmitter, is released from the synaptic junction and enters into the blood circulation. As a result of the increased adrenergic stimulation, the sodium-resorption rises and therewith also the retention of water from the kidney.

The increased circulating plasmacatecholamins lead to a stimulation of the juxtaglomerulous apparatus and to an increased release of renin. Even a decrease of arterial blood pressure or a diminished plasma level of sodium already lead to a stimulation of renin release.

By catalytic cleavage of a protein chain renin causes the release of angiotensin I from angiotensinogen. Angiotensin I as such is transformed to angiotensin II by the angiotensin-converting-enzyme (ACE). This initiates a strong vasoconstriction by activating specific angiotensin II-receptor, increasing the peripheral resistance in case of lowered heart time volume and thereby increasing arterial blood pressure.

Furthermore—at the receptors of the central nervous system—angiotensin II exhibits the function of a neurotransmitter stimulating the thirst center and thereby leading to an increased water take-up. Additionally angiotensin II stimulates the release of the steroid hormone aldosteron from the adrenal body resulting in an increased sodium absorption at the expense of potassium.

Thus, the stimulation of the renin-angiotensin-aldosteron system especially leads to two regulatory mechanisms contravening arterial blood pressure reduction: at the one hand the heart's preload is augmented by an increased retention of water and sodium resulting in a higher heartbeat volume in heart insufficiency. On the other hand the increased peripheral resistance contributes to a normalization of arterial blood pressure in case of reduced heart time volume in order to supply the essential organs with sufficient blood circulation.

The renin-angiotensin-aldosteron system therefore enables maintaining a stable blood pressure even under conditions of timely limited physical exhaust or diarrhea, when the blood volume is reduced and blood pressure decreases. However, in certain individuals this regulatory system is overactive, resulting in a blood pressure which is increased to a pathological value. This increased blood pressure can cause blood vessel damages and thus can lead in the long run e.g. to heart diseases or to stroke.

Therefore, the search for a suitable therapeutic to treat pathological hypertension was directed to a biologically active substance acting as an inhibitor of the renin-angiotensin-aldosteron system.

After having defined the desired physiological property of this substance—namely the inhibition of the renin-angiotensin-aldosteron-system—the next essential step for a successful investigation was the selection of a suitable reference organism expressing a substance with this desired property in adaptation to its natural way of life.

Within this process it was possible to refer back to reports dated from the 60's and describing the collapse of Brazilian farm workers, which were caused by bites of the pit viper Bothrops jararaca. A British team of researchers later revealed that these symptoms were caused by peptides within the snake venom. These peptides were called Bradykinin potentiating peptides (BPP) since they stimulated the effect of the kinin bradykinin.

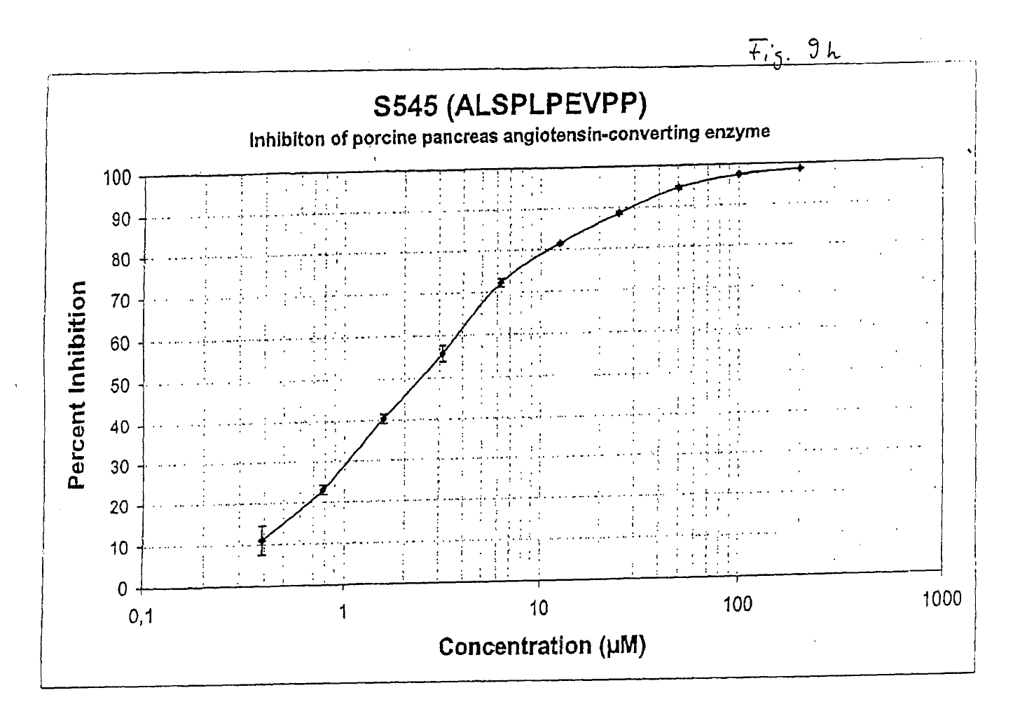

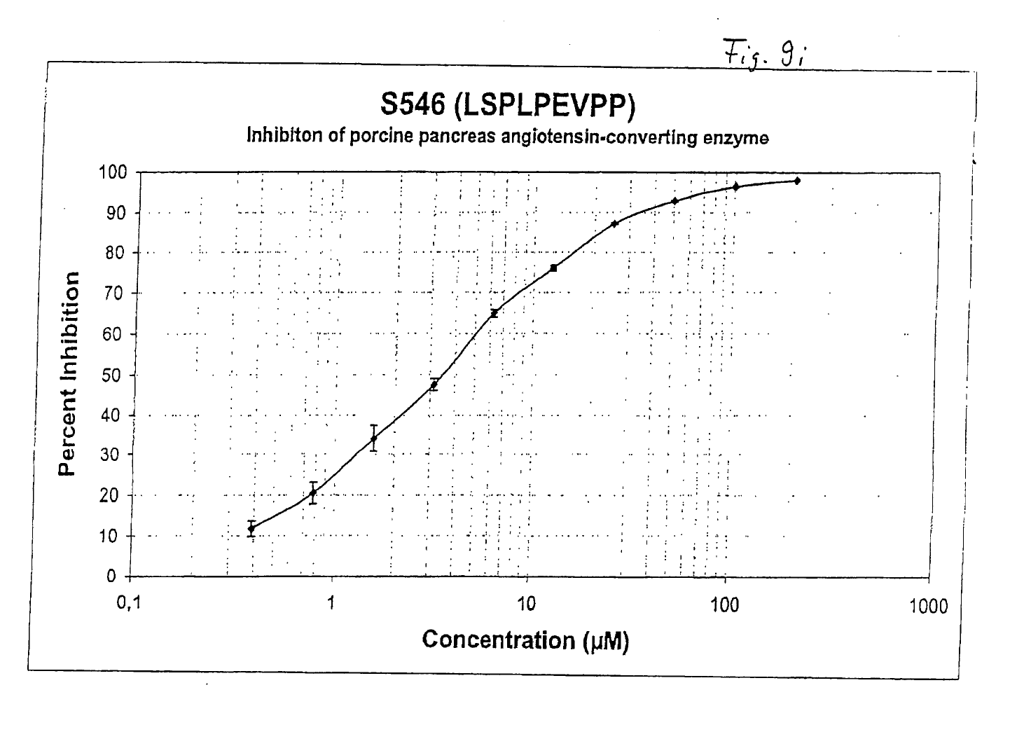

BPPs potentiate the body own bradykinin's vasodilatory and natriuretic effect and thereby lead to a reduced blood pressure (FIG. 8). This effect mainly is due to an inhibition of the angiotensin-converting enzyme (ACE), resulting in a termination of the production of angiotensin II, i.e. a substance essentially involved in causing hypertension. Since ACE catalyses the hydrolytic decomposition of bradykinin, the inhibition of ACE is accopmanied with a prolonged half-life of the otherwise quickly decomposed bradykinin and thus with a potentiated dilatory effect of bradykinin.

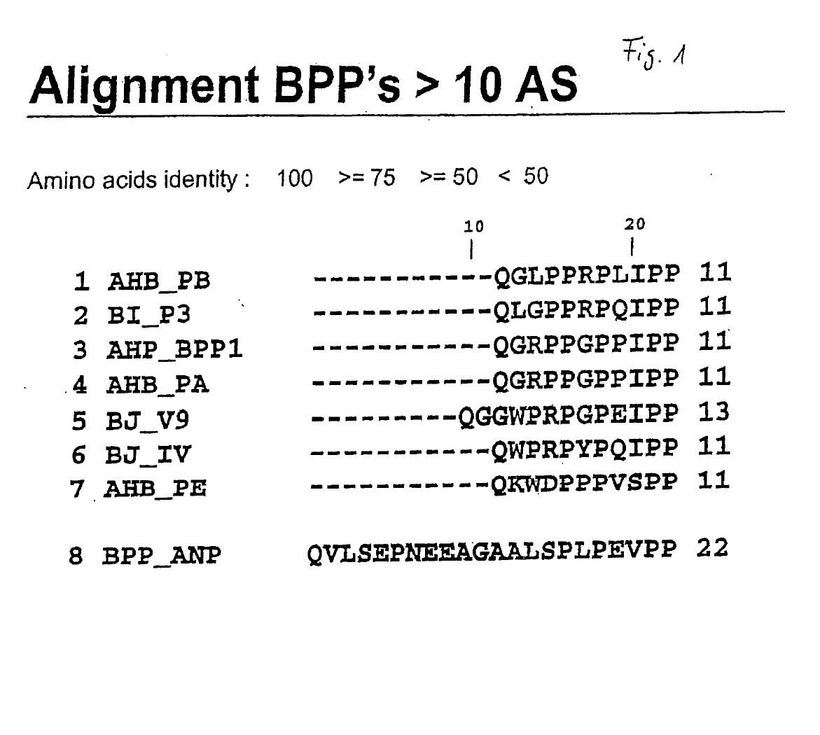

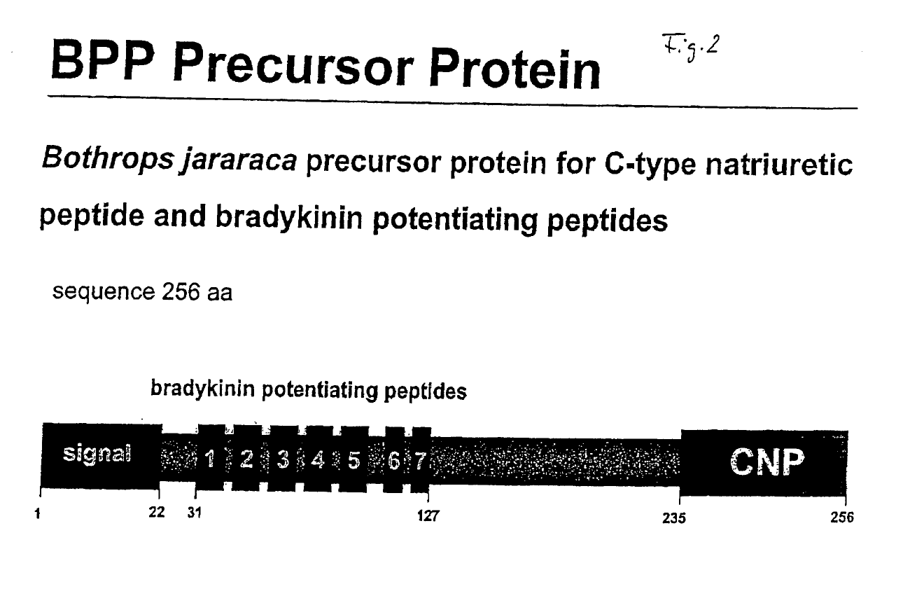

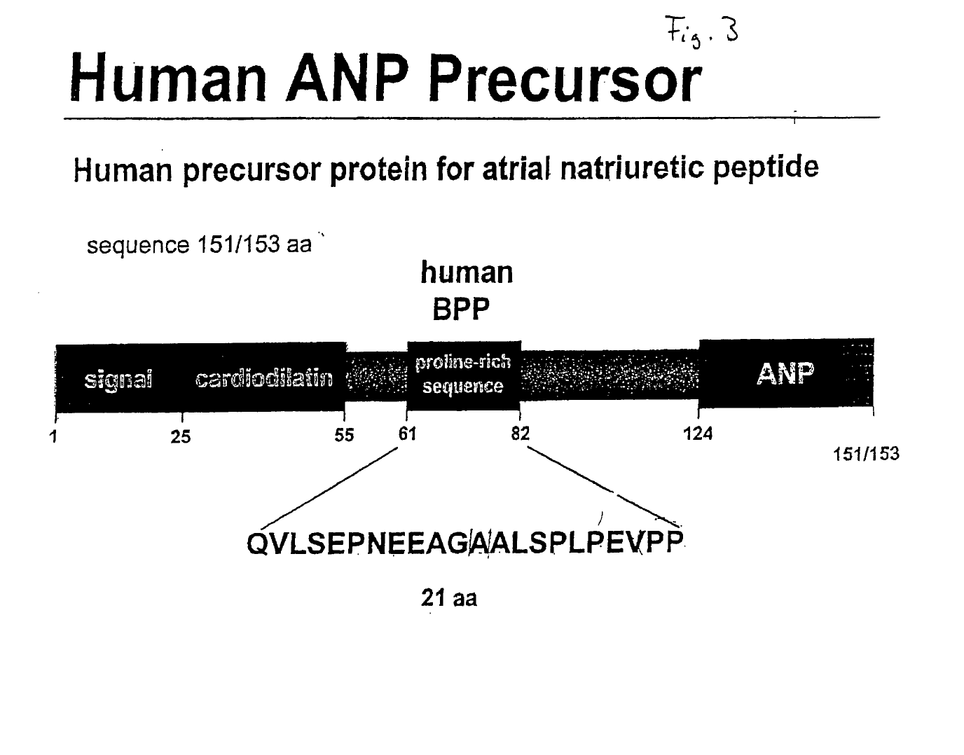

In the following years sequence- and structure analyses of peptides isolated from other snake venoms with bradykinin potentiating effects (e.g. from Bothrops insularis, Bothrops jararaca, Agkistrodon halys blomhoffi and Agkistrodon halys pallas; FIG. 1) lead to the deduction of the following general structural features of BPPs:

All known BPPs are oligopeptides with a maximal length of about 13 amino acid residues with a proline rich sequence and the cyclic amino acid pyroglutamate at the N-terminus, which genetically is encoded as glutamine. With only a few exceptions, at the C-terminus a three-amino acid peptide isolycyl-prolyl-proline—very rarely a seryl-prolyl-proline—can be found with a free carbonic acid group —COOH constituting the terminus.

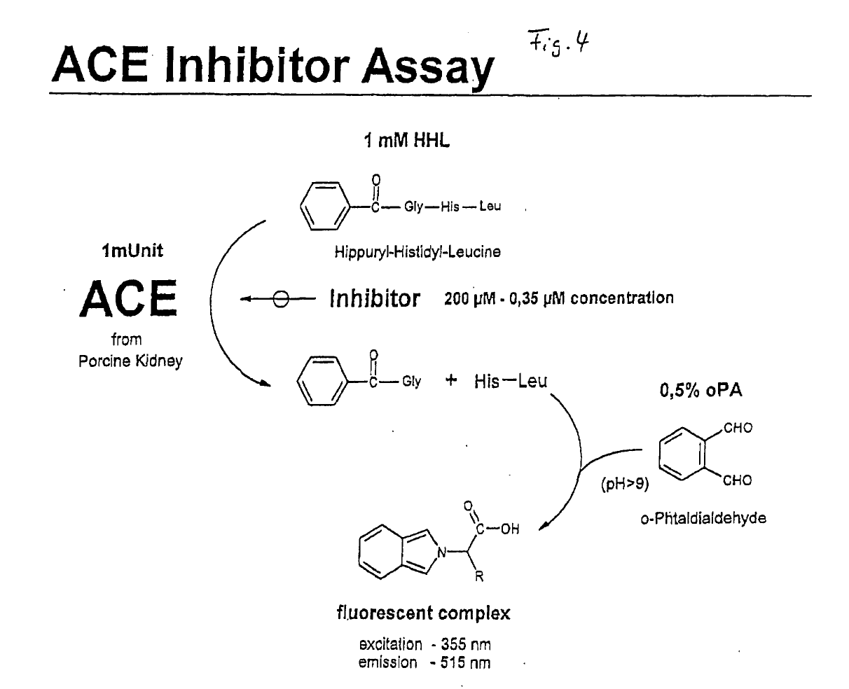

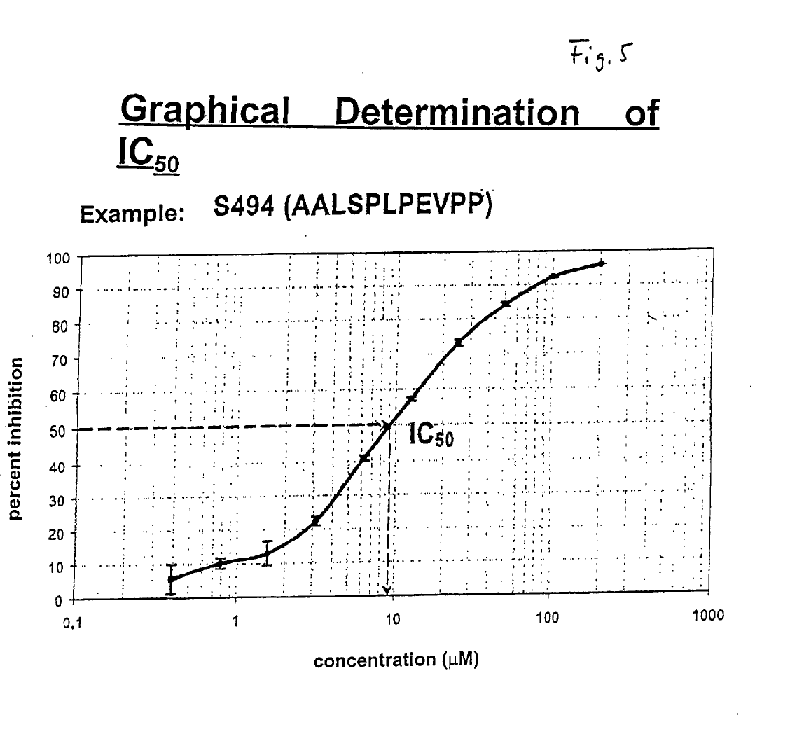

In a further step of comparative data base analyses combined with a comparative literature survey it was found, that snake- and lizard-venoms are both encoded in a tandem orientation within a precurser sequence, containing at its C-terminus a peptide with natriuretic effect. For example, the sequence 256 aa of Bothrops jararaca is a BPP-precurser protein, which encodes N-terminally a BPP following a signal sequence, whereas the natriuretic peptide of the C-type follows in C-terminal direction (CNP; FIG. 2).

In particular, these conclusions are based on the following ideas and considerations:

High throughput sequencing in a cDNA-library derived from the salivary glands of Heloderma horridum horridum led to the following cDNA-sequence:

| helo_all.0.630 (natriuretic peptide precursor) | | |
|---|---|---|
| 1 | cgttcccgga ggatccagca cagactgtgg tgggcggcag cacaaagatg | (SEQ. ID No 1) |
| 51 | aatcccagac tcgcctgctc cacttggctc ccgctgctcc tggtgctgtt | |
| 101 | cactctcgat caggggaggg ccaatccagt ggaaagaggc caggaatatc | |
| 151 | ggtccctgtc taaacggttc gacgacgatt ctaggaaact gatcttagag | |
| 201 | ccaagagcct ctgaggaaaa tggtcctcca tatcaaccct tagtcccaag | |
| 251 | agcttccgac gaaaatgttc ctcctgcatt tgtgccctta gtcccgagag | |
| 301 | cttccgacga aaatgttcct cctcctcctc tgcaaatgcc cttaatcccg | |
| 351 | agagcttccg atgaaaatgt tcctcctcct cctctgcaaa tgcccttaat | |
| 401 | cccgagagcc tccgagcaaa aaggtcctcc atttaatcct ccgccatttg | |
| 451 | tggactacga gccaagagcc gccaatgaaa atgctcttcg gaaactcatc | |
| 501 | aagcgctctt tcgagaggtc cccagggagg aacaaaaggc tcagtcccgg | |
| 551 | agacggctgc tttggtcaga aaattgaccg gatcggagcc gtgagtggga | |
| 601 | tgggatgtaa tagtgtaagc tcacagggga aaaaataata gaaggggatg | |
| 651 | cctgaatcct caaaaaatcc atataattga agcaaaggtc tgcaaggttg | |
| 701 | tattttaaaa aataaaaaat actcctgcca actgaa | |

A comparison of this cDNA-sequence with sequences in public data bases allowed for the following result:

!!SEQUENCE_LIST 1.0

BLASTP 1.4.8 [1-Feb-95] [Build 15:31:04 Feb 10 1997]

Reference: Altschul, Stephen F., Warren Gish, Webb Miller, Eugene W. Myers, and David J. Lipman (1990). Basic local alignment search tool. J. Mol. Biol.

Query=/home/izm/sg37645/helo630.pep

(196 letters)

Database: swplus

239,439 sequences; 76,635,939 total letters.

Smallest

Sum

High Probability

Sequences producing High-scoring Segment Pairs: Score P(N) N

. . .

SW:ANF_CHICK!P18908 gallus gallus (chicken). atrial nat . . . 96 3.7e-07 2

SW:SSGP_VOLCA!P21997 volvox carteri. sulfated surface g . . . 110 1.3e-06 1

SP_OV:P79799!P79799 micrurus corallinus. natriuretic pe . . . 103 6.7e-06 1

SW:ANF_HUMAN!P01160 homo sapiens (human). atrial natriu . . . 82 8.3e-06 3

SP_HUM:Q13766!Q13766 homo sapiens (human). atrial natri . . . 82 1.1e-05 3

SW:ANFB_RAT!P13205 rattus norvegicus (rat). brain natri . . . 99 2.1e-05 1

SP_PL:P93797!P93797 volvox carteri. pherophorin-s precu . . . 100 3.5e-05 1

SW:ANFV_ANGJA!P22642 anguilla japonica (japanese eel) . . . 88 5.4e-05 1

SW:NO75_SOYBN!P08297 glycine max (soybean). early nodul . . . 83 5.9e-05 2

SW:ANFC_HUMAN!P23582 homo sapiens (human). c-type natri . . . 82 05 2

This result led to the conclusion, that the found cDNA-sequence from H. horridum horridum encodes a precursor protein of a natriuretic peptide. Furthermore, it was known in literature, that natriuretic peptides can be found in snake venoms (Schweitz H, Vigne P, Moinier D, Frelin C, Lazdunski M. A new member of the natriuretic peptide family is present in the venom of the green mamba Dendroaspis angusticeps; J Biol Chem. Jul. 15, 1992; 267 (20):13928-32.), of which the precursor sequences also code for BPPs (Murayama N, Hayashi M A, Ohi H, Ferreira L A, Hermann W, Saito H, Fujita Y, Higuchi S, Fernandes B L, Yamane T, de Camargo A C. Cloning and sequence analysis of a Bothrops jararaca cDNA encoding a precursor of seven bradykinin-potentiating peptides and a C-type natriuretic peptide. Proc Natl Acad Sci USA. Feb. 18, 1997;94(4):1189-93).

The following sequence shows the prepro-form of a precursor of a natriuretic peptide from Heloderma horridum horridum.

helo_all.0.630 (natriuretic peptide precursor)

(SEQ. ID. No 2)

signal peptide

| | |
|---|---|
| 1 | MNPRLACSTW LPLLLVLFTL DQGRANPVER GQEYRSLSKR |
| | FDDDSRKLIL |
| 51 | EPRASEENGP PYQPLVPRAS DENVPPAFVP LVPRASDENV |
| | PPPPLQMPLI |
| 101 | PRASDENVPP PPLQMPLIPR ASEQKGPPFN PPPFVDYEPR |
| | AANENALRKL |
| 151 | IKRSFERSPG RNKRLSPGDG CFGQKIDRIG AVSGMGCNSV |
| | SSQGKK |

Natriuretic Peptide

This sequence indicates, that the region located N-terminally from the potential natriuretic peptide (determined by homology) contains sequence elements, which are very similar or identical to each other. It is assumed that—in analogy to known precursor-sequences of natriuretic peptides and BPPs (bradykinin-potentiating peptides) from snakes—these sequence sections encode for peptides, which potentiate the physiological effects of bradykinin and—as BPPs from snakes—also inhibit ACE (angiotensin-converting enzyme).

In order to examine, if these peptides constitute ACE-inhibitors, two peptides were synthesized (S682 and S683—sequence see below). The C-terminus was selected to be prolyl-proline as it is known from BPPs from snake venoms. In the precursor-protein three sequence sections are identical. This amino acid-sequence was chosen for a peptide (S683). The second peptide (S682) comprises the same sequence. However, N-terminally it is extended with five additional amino acids. In this peptide, the N-terminal glutamin was substituted with pyroglutamate. Pyroglutamate is encoded as glutamin. It is constituted by enzymatic modification. Since all known BPPs found in snakes contain pyroglutamate as N-terminus, this was also assumed for Heloderma.

The two Heloderma-peptides were tested for their ACE-inhibitory activity (same assay like in human BPPs; see below).

The IC₅₀-values for ACE-inhibitors derived from pig kidney are presented in the following table (Tab.6):

TABLE 6

| Inhibitor | Structure | IC₅₀-value | |
|---|---|---|---|
| Captopril | 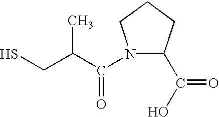 | 0.0014 | μM |
| BPP9a | pGlu-WPRPQIPP | 0.097 | μM |
| S682 | pGlu-MPLIPRASDENVPP | 150 | μM |
| (SEQ: ID. No 3) | | | |
| S683 | PRASDENVPP | 65 | μM |
| (SEQ. ID. No 4) | | | |

With these findings as a starting point, the (pro)precursor-sequences of human natriuretic peptides were analyzed with respect to these general structural characteristics. Therewith, in the (pro)precursor-protein of the atrial natriuretic hormone (ANP), it was possible to identify a proline-rich sequence motif, displaying the same proline pattern as found in snake-BPP, namely a C-terminal prolyl-proline and two additional proline residues in N-terminal direction:

ANF_Human Atrial Natriuretic Factor Precursor (ANF)

sequence 153 aa; 16708 MW

| | | |
|---|---|---|
| signal | 1 . . . 25 | signal peptide |
| peptide | 26 . . . 55 | cardiodilatin-related peptide(CDP) |
| peptide | 73 . . . 82 | human BPP |
| peptide | 124 . . . 151 | atrial natriuretic peptide (ANP) |
| disulfid | 130 . . . 146 | by similarity |
| variant | 152 . . . 153 | missing (in one of the two genes) |

| | |
|---|---|
| 1 | MSSFSTTTVS FLLLLAFQLL GQTRANPMYN AVSNADLMDF |
| | KNLLDHLEEK |
| 51 | MPLEDEVVPP QVLSEPNEEA GAALSPLPEV PPWTGEVSPA |
| | QRDGGALGRG |
| 101 | PWDSSDRSAL LKSKLRALLT APRSLRRSSC FGGRMDRIGA |
| | QSGLGCNSFR |
| 151 | YRR |

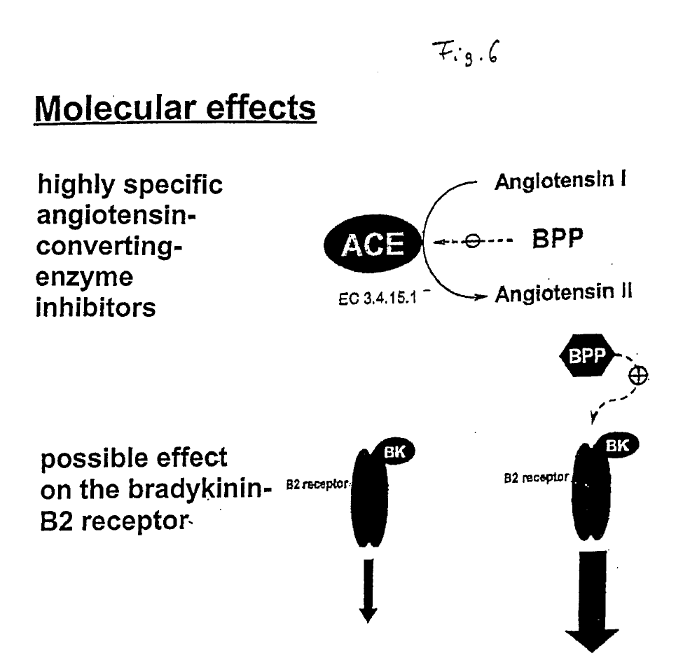

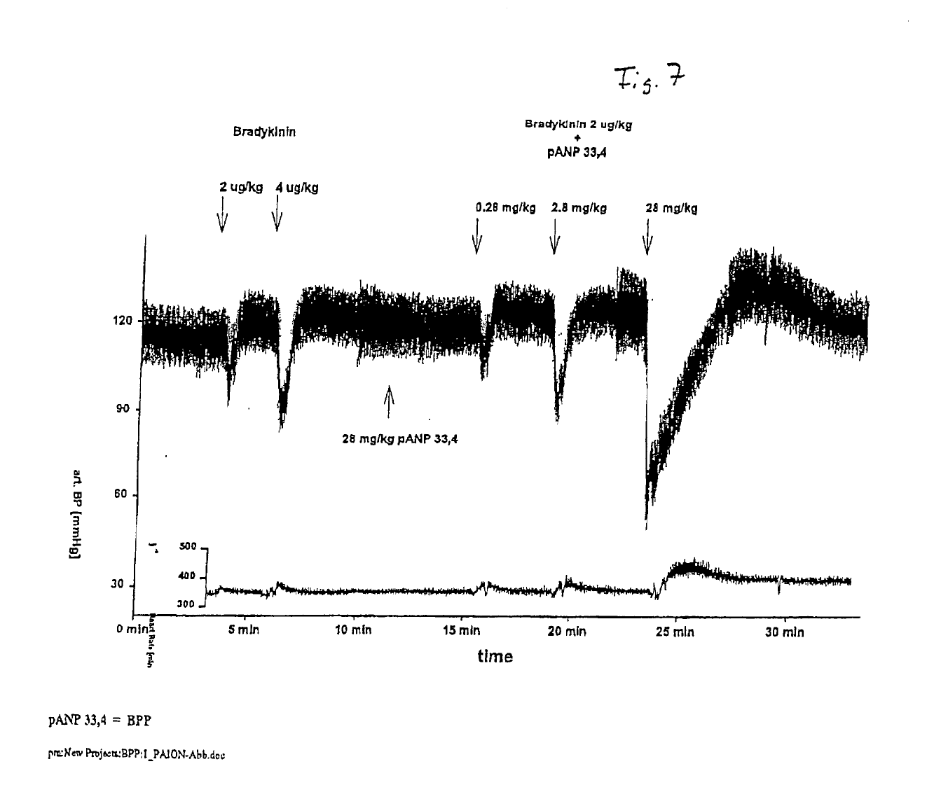

An overview for the human ANP-proprecursor-protein is shown in FIG. 3. After the dissection of the signal sequence, a precursor-sequence pANP remains, being shortened by 25 amino acid residues.

In this short sequence section the characteristic proline pattern of snake venoms with over 10 amino acids can be confirmed:

Amino acids identity: 100 >= 75 >= 50 < 50

| | | | |
|---|---|---|---|
| 1 | AHB_PB | -----------QGLPPRPLIPP | 11 |
| 2 | BI_P3 | -----------QLGPPRPQIPP | 11 |
| 3 | AHP_BPP1 | -----------QGRPPGPPIPP | 11 |
| 4 | AHB_PA | -----------QGRPPGPPIPP | 11 |
| 5 | BJ_V9 | ---------QGGWPRPGPEIPP | 13 |
| 6 | BJ_IV | -----------QWPRPYPQIPP | 11 |
| 7 | AHB_PE | -----------QKWDPPPVSPP | 11 |
| 8 | BPP_ANP | QVLSEPNEEAGAALSPLPEVPP | 22 |

Numbers 1-7 are BPPs derived from snake venoms, number 8 is a 22 amino acid sequence section from human proANP; comprising AS-positions 61 to 82.

Starting from the detected amino acid sequences of human pANP (precursor ANP), peptides all containing prolyl-proline at the C-terminus were synthesized. These peptides vary in sequence length from 7 to 15 amino acids. The sequences of these peptides are shown in table 1.

TABLE 1

| Peptide | Sequence | IC₅₀ |
|---|---|---|
| S541 | EEAGAALSPLPEVPP | 48 μM |
| (SEQ. ID No 5) | | |
| S542 | EAGAALSPLPEVPP | 38 μM |
| (SEQ. ID No 6) | | |
| S543 | AGAALSPLPEVPP | 25 μM |
| (SEQ. ID No 7) | | |
| S544 | GAALSPLPEVPP | 29 μM |
| (SEQ. ID No 8) | | |
| S494 | AALSPLPEVPP | 9 μM |
| (SEQ. ID No 9) | | |
| S545 | ALSPLPEVPP | 2.4 μM |
| (SEQ. ID No 10) | | |
| S546 | LSPLPEVPP | 3.5 μM |
| (SEQ. ID No 11) | | |
| S547 | SPLPEVPP | 27 μM |
| (SEQ. ID No 12) | | |
| S548 | PLPEVPP | 21 μM |
| (SEQ. ID No 13) | | |

Hereby, sequence lengths of 15 amino acids were not exceeded, since longer sequences potentially form secondary structures, which reduce activity. This was already shown in experiments using snake-BPPs. As a control a peptide of 22 amino acid residues was synthesized.

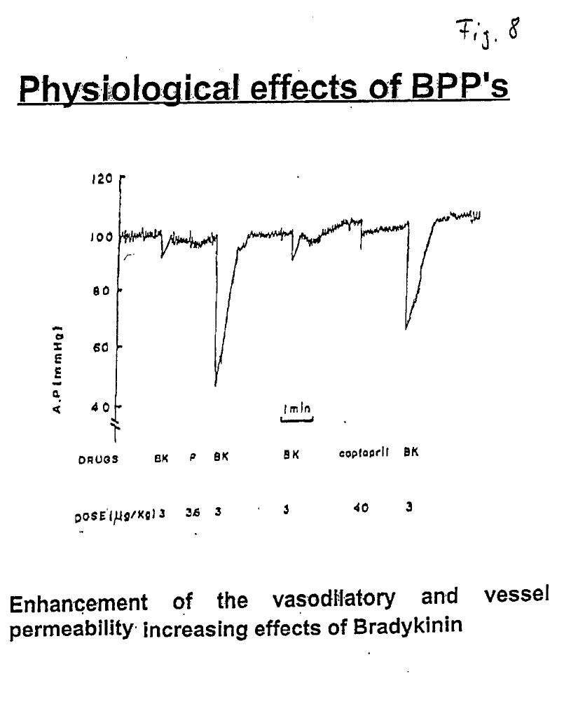

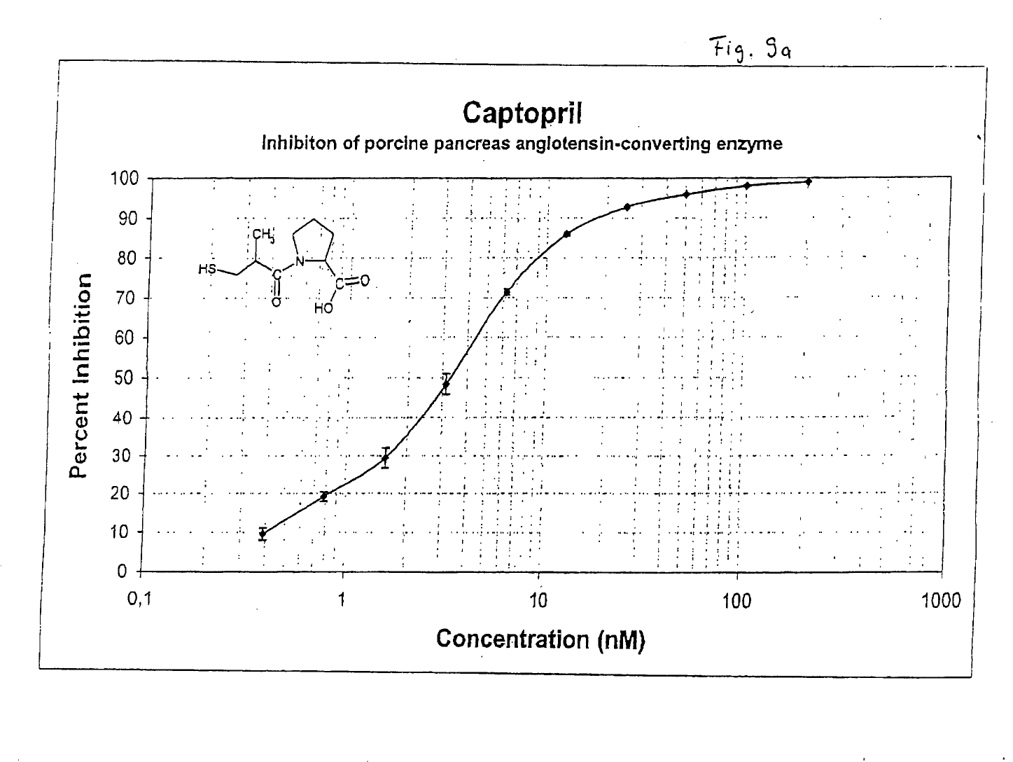

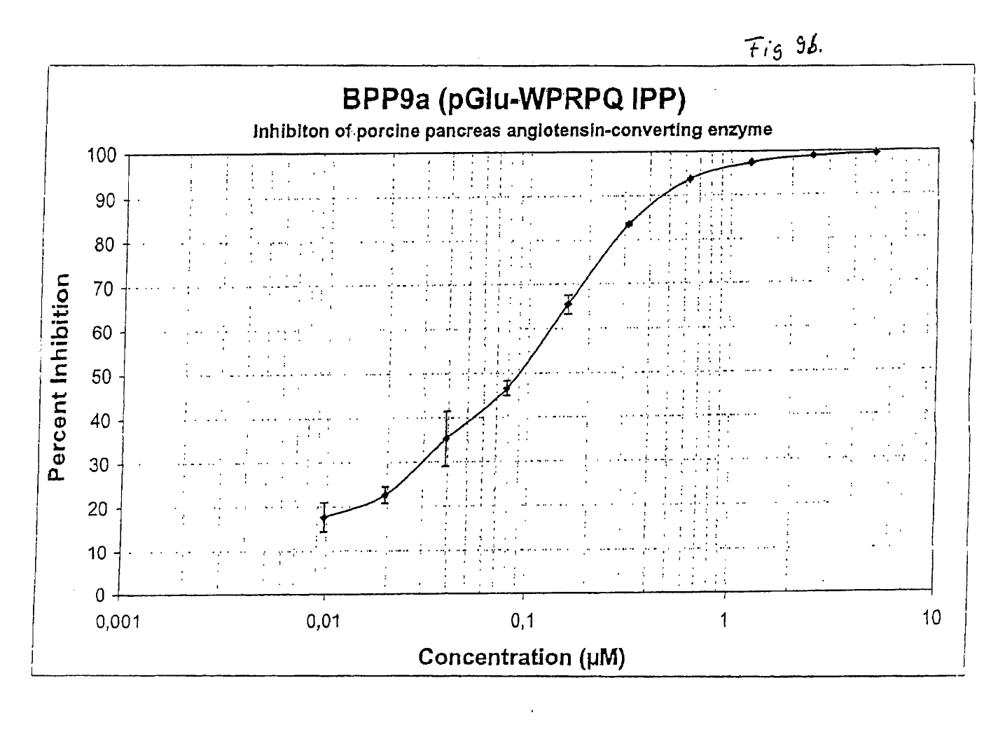

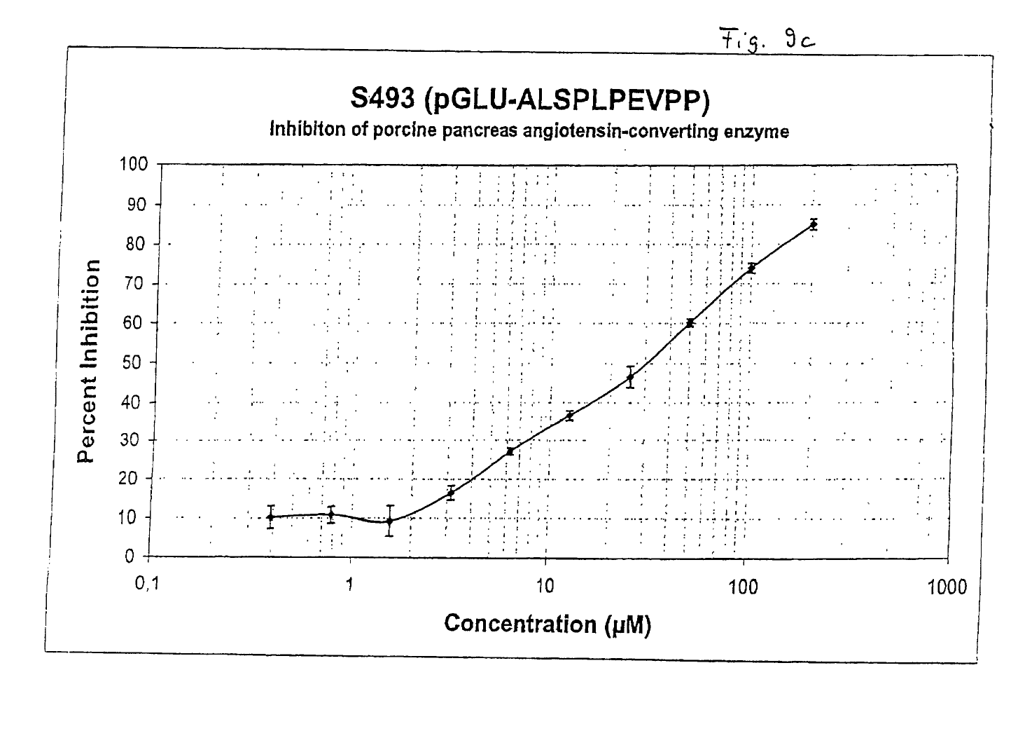

These peptides were investigated for their inhibitory effect using an in vitro-ACE-inhibitor-assay with an angiotensin-converting-enzyme derived from pig kidney. Hippuryl-histidyl-leucin was used as a substrate. A schematic presentation of the assay is shown in FIG. 4; an exemplary graphical determination of the IC₅₀-values is subject of FIG. 5. A concrete experimental description is given below. The respective IC₅₀-values are presented in Tab.1.

In addition to these peptides (synthesized and purchased from Biosyntan), BPP9a from Bothrops jararaca (Sigma) and Captopril (Sigma)—the first synthetic ACE-inhibitor developed on the basis of snake-BPP research—were analyzed for their property to inhibit ACE.

TABLE 2

| Inhibitor | Structure | IC₅₀ | |
|---|---|---|---|
| Captopril | 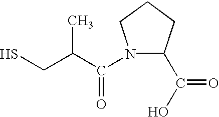 | 0.0014 | μM |
| BPP9a | Pyr-WPRPQIPP | 0.097 | μM |
| S492 | pGlu-VLSEPNEEAGAALSPLPEVPP | >300 | μM |
| (SEQ. ID. | | | |
| No 14) | | | |
| S493 | pGlu-ALSPLPEVPP | 20 | μM |
| (SEQ. ID. | | | |
| No. 15) | | | |
| S494 | AALSPLPEVPP | 9 | μM |

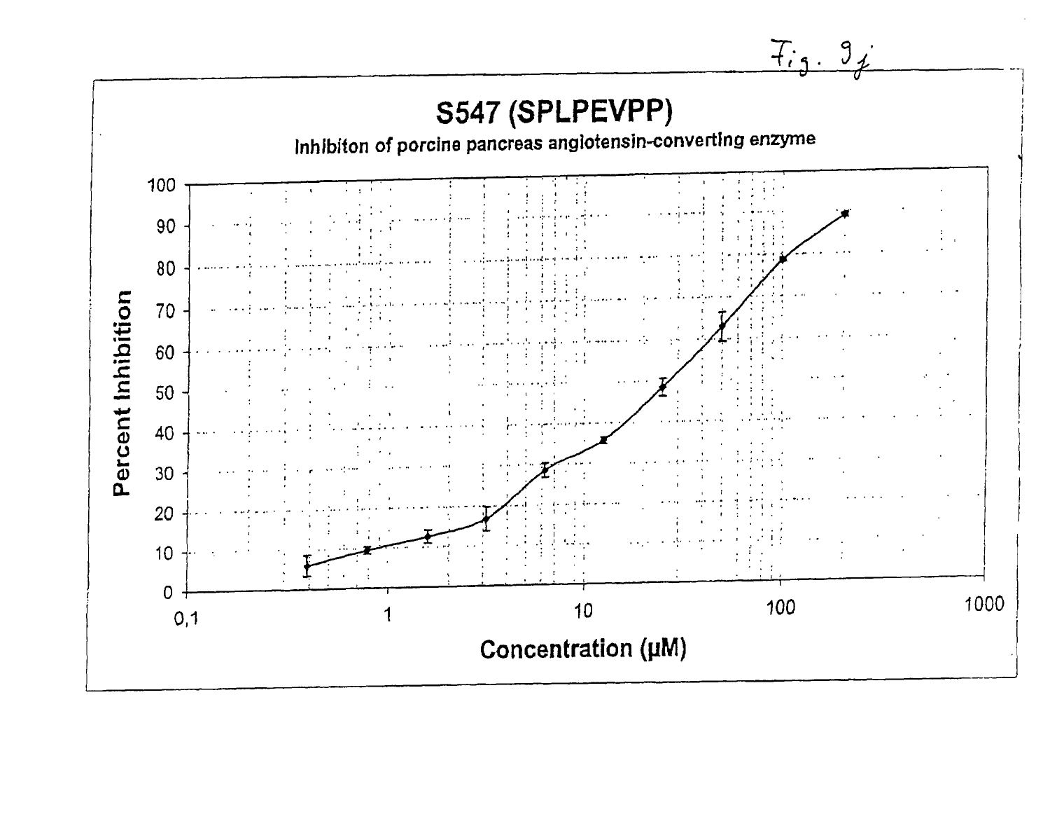

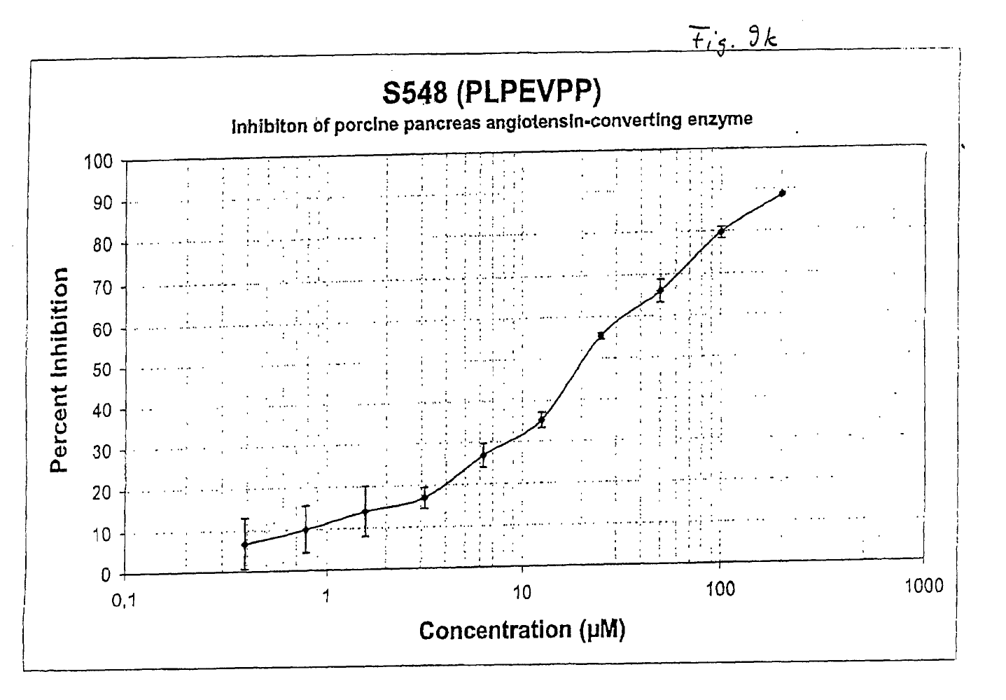

It can be summarized, that the decapeptide S545 already at a concentration of 2,4 μM exhibits an inhibitory effect with half maximal value. Further shortening of this peptide to 9, 8 or 7 amino acids led to an increase of the IC₅₀-values. The same occurs with extending the peptide to 15 amino acids. For example, the IC₅₀-value for BPP S541 is 48 μM. Extending the peptide to 22 amino acids including a transformation of the N-terminus to pyroglutamate in-vitro resulted in an IC₅₀-value of over 300 μM and thus, nearly to inactivity (Tab. 2). These results are shown in the tables 5a-5k and in FIGS. 9a-9k.

TABLE 5a
ACE Inhibitor Assay Captopril

| | Control 1 | | | | | | | | | | | Control 2 |
|---|---|---|---|---|---|---|---|---|---|---|---|---|
| | 200 μM | | | | | | | | | | | (no |
| | Inhibitor | 200 nM | 100 nM | 50 nM | 25 nM | 12.5 nM | 6.25 nM | 3.125 nM | 1.5625 nM | 0.78 nm | 0.39 nM | Inhibitor) |
| A | 4040 | 10533 | 17725 | 30454 | 57450 | 100688 | 189881 | 361035 | 570796 | 647369 | 719067 | 870140 |
| | 100% | 99.33% | 98.93% | 97.54% | 94.05% | 83.96% | 64.3% | 48.26% | 26.86% | 24.38% | 17.99% | 0% |
| B | 4108 | 10605 | 16048 | 28671 | 52258 | 88033 | 178042 | 320986 | 516330 | 586886 | 666248 | 885634 |
| | 100% | 99.44% | 98.94% | 97.35% | 93.5% | 83.75% | 63.32% | 43.47% | 35.44% | 24.17% | 17.32% | 0% |
| C | 5069 | 10761 | 15702 | 28448 | 49833 | 91942 | 170639 | 318112 | 478116 | 604617 | 687659 | 832432 |
| | 100% | 99.44% | 98.91% | 97.4% | 93.43% | 83.22% | 64.08% | 48.35% | 31.2% | 22.97% | 14.76% | 0% |
| D | 4346 | 11520 | 16219 | 29108 | 52493 | 115956 | 175748 | 305395 | 458934 | 605543 | 680014 | 758087 |
| | 100% | 99.44% | 98.79% | 97.38% | 93.82% | 83.09% | 69.83% | 46.37% | 47.3% | 18.85% | 7.86% | 0% |

Control 1: without enzyme
Control 2: without inhibitor

TABLE 5b
ACE Inhibitor Assay
Peptid BPP9a (pGlu-WPRPQIPP)

| | Control 1 | | | | | | | | | | | Control 2 |
|---|---|---|---|---|---|---|---|---|---|---|---|---|
| | 200 μM | | | | 0.625 | | | | | | | (no |
| | Inhibitor | 5 μM | 2.5 μM | 1.25 μM | μM | 0.3125 μM | 0.15625 μM | 0.078 μM | 0.039 μM | 0.0195 μM | 0.0097 μM | Inhibitor) |
| A | 5151 | 10561 | 13979 | 25833 | 55616 | 141700 | 309352 | 446174 | 628809 | 649942 | 704452 | 832224 |
| | 100% | 99.33% | 98.93% | 97.54% | 94.05% | 83.96% | 64.3% | 48.26% | 26.86% | 24.38% | 17.99% | 0% |
| B | 6145 | 9634 | 13903 | 27485 | 60333 | 143514 | 317736 | 487053 | 555538 | 651703 | 710140 | 860834 |
| | 100% | 99.44% | 98.94% | 97.35% | 93.5% | 83.75% | 63.32% | 43.47% | 35.44% | 24.17% | 17.32% | 0% |
| C | 3976 | 9626 | 14148 | 26994 | 60929 | 147997 | 311280 | 445495 | 591749 | 661931 | 732003 | 887052 |
| | 100% | 99.44% | 98.91% | 97.4% | 93.43% | 83.22% | 64.08% | 48.35% | 31.2% | 22.97% | 14.76% | 0% |
| D | 4128 | 9656 | 15119 | 27156 | 57514 | 149103 | 262186 | 462323 | 454421 | 697146 | 790803 | 936804 |
| | 100% | 99.44% | 98.79% | 97.38% | 93.82% | 83.09% | 69.83% | 46.37% | 47.3% | 18.85% | 7.86% | 0% |

Control 1: without enzyme
Control 2: without inhibitor

TABLE 5c
ACE Inhibitor Assay
Peptid S494 (AALSPLPEVPP)

| | Control 1 | | | | | | | | | | | Control 2 |
|---|---|---|---|---|---|---|---|---|---|---|---|---|
| | 200 μM | | | | | | | | | 0.78125 | | (no |
| | Inhibitor | 200 μM | 100 μM | 50 μM | 25 μM | 12.5 μM | 6.25 μM | 3.125 μM | 1.5625 μM | μM | 0.39 μM | Inhibitor) |
| A | 4987 | 34738 | 57543 | 108988 | 188693 | 308116 | 433344 | 556345 | 606137 | 633619 | 659968 | 703126 |
| | 100% | 95.82% | 92.64% | 85.48% | 74.38% | 57.74% | 40.3% | 23.17% | 16.23% | 12.41% | 8.74% | 0% |
| B | 5933 | 34342 | 59914 | 119135 | 193365 | 312590 | 420434 | 563277 | 602329 | 643151 | 704319 | 688102 |
| | 100% | 95.87% | 92.31% | 84.06% | 73.73% | 57.12% | 42.1% | 22.2% | 16.77% | 11.08% | 2.56% | 0% |
| C | 4138 | 34053 | 62551 | 120732 | 203903 | 306320 | 435365 | 547546 | 645065 | 655606 | 727603 | 630039 |
| | 100% | 95.91% | 91.94% | 83.84% | 72.26% | 57.99% | 40.02% | 24.4% | 10.82% | 9.35% | −0.67% | 0% |
| D | 3784 | 34364 | 64312 | 124777 | 204201 | 319249 | 427540 | 579178 | 664903 | 667363 | 762908 | 666287 |
| | 100% | 95.87% | 91.7% | 83.28% | 72.21% | 56.19% | 41.11% | 20% | 8.05% | 7.71% | −5.59% | 0% |

Control 1: without enzyme
Control 2: without inhibitor

TABLE 5d
ACE Inhibitor Assay
Peptid S541 (EEAGAALSPLPEVPP)

| | Control 1 | | | | | | | | | | | Control 2 |
|---|---|---|---|---|---|---|---|---|---|---|---|---|
| | 200 μM | | | | | | | | | 0.78125 | | (no |
| | Inhibitor | 200 μM | 100 μM | 50 μM | 25 μM | 12.5 μM | 6.25 μM | 3.125 μM | 1.5625 μM | μM | 0.39 μM | inhibitor) |
| A | 4164 | 215674 | 351975 | 490677 | 661658 | 781107 | 879836 | 859457 | 946248 | 943566 | 967573 | 1018550 |
| | 100% | 78.25% | 64.21% | 50.07% | 32.32% | 20.02% | 9.85% | 11.95% | 3.02% | 3.3% | 0.82% | 0% |
| B | 3930 | 208164 | 346560 | 480002 | 639406 | 788769 | 836560 | 803411 | 904012 | 907185 | 955811 | 1010820 |
| | 100% | 79.02% | 64.77% | 51.02% | 34.61% | 21.5% | 14.31% | 17.72% | 7.36% | 7.04% | 2.03% | 0% |
| C | 5051 | 201991 | 319033 | 474783 | 631484 | 769930 | 809014 | 810684 | 844202 | 888487 | 921346 | 990200 |
| | 100% | 79.86% | 67.6% | 51.57% | 35.43% | 21.17% | 17.15% | 16.97% | 13.52% | 8.96% | 5.58% | 0% |
| D | 4713 | 204112 | 313789 | 474901 | 614518 | 757658 | 817520 | 810188 | 857232 | 903844 | 917688 | 990200 |
| | 100% | 79.44% | 68.17% | 51.55% | 37.18% | 22.44% | 16.27% | 17.03% | 12.18% | 7.38% | 5.96% | 0% |

Control 1: without enzyme
Control 2: without inhibitor

TABLE 5e
ACE Inhibitor Assay
Peptid S542 (EAGAALSPLPEVPP)

| | Control 1 | | | | | | | | | | | Control 2 |
|---|---|---|---|---|---|---|---|---|---|---|---|---|
| | 200 μM | | | | | | | | | 0.78125 | | (no |
| | Inhibitor | 200 μM | 100 μM | 50 μM | 25 μM | 12.5 μM | 6.25 μM | 3.125 μM | 1.5625 μM | μM | 0.39 μM | Inhibitor) |
| A | 4971 | 169891 | 274375 | 406539 | 569641 | 709216 | 795040 | 796263 | 794177 | 880680 | 906307 | 956711 |
| | 100% | 82.99% | 72.23% | 58.61% | 41.81% | 27.43% | 18.59% | 18.47% | 18.68% | 9.77% | 7.13% | 0% |
| B | 5947 | 167517 | 273140 | 424530 | 609106 | 719799 | 801078 | 821478 | 794214 | 910838 | 930945 | 961605 |
| | 100% | 83.24% | 72.35% | 56.76% | 37.75% | 26.34% | 17.97% | 15.87% | 18.68% | 6.66% | 4.59% | 0% |
| C | 4098 | 173807 | 278465 | 419325 | 591988 | 744223 | 799975 | 820849 | 874684 | 902555 | 967806 | 949704 |
| | 100% | 82.59% | 71.8% | 57.3% | 39.51% | 23.83% | 18.08% | 15.93% | 10.39% | 7.52% | 0.79% | 0% |
| D | 4162 | 184836 | 286263 | 440093 | 618193 | 765229 | 802324 | 868901 | 882788 | 939597 | 1003456 | 931778 |
| | 100% | 81.45% | 71% | 55.16% | 36.81% | 21.66% | 17.84% | 10.98% | 9.55% | 3.7% | −2.87% | 0% |

Control 1: without enzyme
Control 2: without inhibitor

TABLE 5f
ACE Inhibitor Assay
Peptid S543 (AGAALSPLPEVPP)

| | Control 1 | | | | | | | | | | | Control 2 |
|---|---|---|---|---|---|---|---|---|---|---|---|---|
| | 200 μM | | | | | | | | 1.5625 | 0.78125 | | (no |
| | Inhibitor | 200 μM | 100 μM | 50 μM | 25 μM | 12.5 μM | 6.25 μM | 3.125 μM | μM | μM | 0.39 μM | inhibitor) |
| A | 4454 | 111945 | 219372 | 367216 | 534895 | 700323 | 833135 | 938801 | 980940 | 981909 | 996656 | 988711 |
| | 100% | 89.67% | 79.28% | 64.98% | 48.76% | 32.76% | 19.92% | 9.7% | 5.62% | 5.53% | 4.1% | 0% |
| B | 4945 | 107628 | 211437 | 362416 | 519515 | 683603 | 816251 | 868636 | 923507 | 946496 | 959059 | 982545 |
| | 100% | 90.09% | 80.05% | 65.45% | 50.25% | 34.38% | 21.55% | 16.48% | 11.18% | 8.95% | 7.74% | 0% |
| C | 5785 | 110019 | 206169 | 357767 | 531562 | 714973 | 788199 | 880388 | 888699 | 913444 | 985299 | 1029324 |
| | 100% | 89.95% | 80.56% | 65.9% | 49.09% | 31.35% | 24.26% | 15.35% | 14.54% | 12.15% | 5.2% | 0% |
| D | 5401 | 108841 | 209393 | 358975 | 515233 | 720213 | 773151 | 856283 | 915925 | 925926 | 971597 | 1037369 |
| | 100% | 89.97% | 80.25% | 65.78% | 50.66% | 30.84% | 25.72% | 17.68% | 11.9% | 10.945% | 6.53% | 0% |

Control 1: without enzyme
Control 2: without inhibitor

TABLE 5g
ACE Inhibitor Assay
Peptid S544 (GAALSPLPEVPP)

| | Control 1 | | | | | | | | | | | Control 2 |
|---|---|---|---|---|---|---|---|---|---|---|---|---|
| | 200 μM | | | | | | | | 1.5625 | 0.78125 | | (no |
| | Inhibitor | 200 μM | 100 μM | 50 μM | 25 μM | 12.5 μM | 6.25 μM | 3.125 μM | μM | μM | 0.39 μM | Inhibitor) |
| A | 5025 | 94363 | 158918 | 286592 | 521311 | 684354 | 735049 | 883161 | 930998 | 929364 | 936430 | 915708 |
| | 100% | 90.34% | 83.37% | 69.54% | 44.1% | 26.43% | 20.94% | 4.89% | −0.29% | −0.11% | −0.88% | 0% |
| B | 5291 | 92239 | 154967 | 263679 | 494831 | 668628 | 670407 | 822128 | 864708 | 853374 | 841511 | 924023 |
| | 100% | 90.6% | 83.8% | 72.02% | 46.97% | 28.14% | 27.95% | 11.5% | 6.89% | 8.12% | 9.4% | 0% |
| C | 5999 | 93028 | 152193 | 261020 | 495043 | 658367 | 719205 | 840019 | 811879 | 881105 | 894833 | 910591 |
| | 100% | 90.51% | 84.1% | 72.3% | 46.95% | 29.25% | 22.66% | 9.56% | 12.61% | 5.11% | 3.63% | 0% |
| D | 5575 | 95574 | 156501 | 262784 | 522915 | 643614 | 725919 | 799408 | 787983 | 872351 | 882762 | 908551 |
| | 100% | 90.23% | 83.63% | 72.12% | 43.93% | 30.85% | 21.93% | 13.96% | 15.2% | 6.06% | 4.93% | 0% |

Control 1: without enzyme
Control 2: without inhibitor

TABLE 5h
ACE Inhibitor Assay
Peptid S545 (ALSPLPEVPP)

| | Control 1 | | | | | | | | | | | Control 2 |
|---|---|---|---|---|---|---|---|---|---|---|---|---|
| | 200 μM | | | | | | | | | 0.78125 | | (no |
| | inhibitor | 200 μM | 100 μM | 50 μM | 25 μM | 12.5 μM | 6.25 μM | 3.125 μM | 1.5625 μM | μM | 0.39 μM | inhibitor) |
| A | 5197 | 18937 | 28568 | 48696 | 104078 | 177772 | 250076 | 392263 | 551569 | 692816 | 774338 | 906488 |
| | 100% | 98.5% | 97.45% | 95.27% | 89.27% | 81.29% | 73.46% | 58.06% | 40.8% | 25.5% | 16.67% | 0% |
| B | 6704 | 17677 | 28035 | 53158 | 108586 | 174037 | 251538 | 405396 | 541353 | 726277 | 811412 | 931605 |
| | 100% | 98.6% | 97.51% | 94.79% | 88.78% | 81.69% | 73.3% | 56.63% | 41.9% | 21.88% | 12.66% | 0% |
| C | 4220 | 17238 | 29130 | 63009 | 113328 | 177324 | 279216 | 408086 | 551184 | 720995 | 841911 | 955984 |
| | 100% | 98.68% | 97.39% | 93.72% | 88.27% | 81.34% | 70.3% | 56.36% | 40.85% | 22.45% | 9.36% | 0% |
| D | 4156 | 18084 | 30237 | 54447 | 113458 | 175378 | 263340 | 447790 | 577933 | 719037 | 876502 | 973486 |
| | 100% | 98.59% | 97.27% | 94.65% | 88.26% | 81.55% | 72.02% | 52.05% | 37.95% | 22.66% | 5.61% | 0% |

Control 1: without enzyme
Control 2: without inhibitor

TABLE 5i
ACE Inhibitor Assay
Peptid S546 (LSPLPEVPP)

| | Control 1 | | | | | | | | | | | Control 2 |
|---|---|---|---|---|---|---|---|---|---|---|---|---|
| | 200 μM | | | | | | | | | 0.78125 | | (no |
| | Inhibitor | 200 μM | 100 μM | 50 μM | 25 μM | 12.5 μM | 6.25 μM | 3.125 μM | 1.5625 μM | μM | 0.39 μM | Inhibitor) |
| A | 4523 | 22511 | 39445 | 73176 | 124101 | 226385 | 336576 | 495814 | 657580 | 763649 | 826280 | 907909 |
| | 100% | 98.06% | 96.17% | 92.41% | 86.74% | 75.35% | 63.07% | 45.32% | 27.32% | 15.5% | 8.53% | 0% |
| B | 4925 | 22822 | 37664 | 69502 | 116421 | 225714 | 308510 | 483121 | 591636 | 719861 | 803315 | 897696 |
| | 100% | 98.03% | 96.37% | 92.82% | 87.60% | 75.43% | 66.2% | 46.75% | 34.66% | 20.38% | 11.08% | 0% |
| C | 5777 | 23110 | 36789 | 65379 | 119382 | 208268 | 312423 | 472396 | 572728 | 687713 | 773719 | 887580 |
| | 100% | 97.99% | 96.47% | 93.28% | 87.27% | 77.32% | 65.77% | 47.95% | 36.77% | 23.96% | 14.38% | 0% |
| D | 5325 | 24700 | 37490 | 67861 | 118514 | 221839 | 322970 | 452574 | 566984 | 702450 | 787391 | 917217 |
| | 100% | 97.82% | 96.39% | 93.01% | 87.36% | 75.86% | 64.59% | 50.15% | 37.41% | 22.32% | 12.86% | 0% |

Control 1: without enzyme
Control 2: without inhibitor

TABLE 5j
ACE Inhibitor Assay
Peptid S547 (SPLPEVPP)

| | Control 1 | | | | | | | | | | | Control 2 |
|---|---|---|---|---|---|---|---|---|---|---|---|---|
| | 200 μM | | | | | | | | | 0.78125 | | (no |
| | inhibitor | 200 μM | 100 μM | 50 μM | 25 μM | 12.5 μM | 6.25 μM | 3.125 μM | 1.5625 μM | μM | 0.39 μM | inhibitor) |
| A | 6089 | 113488 | 214324 | 346051 | 501835 | 660883 | 715138 | 814056 | 900439 | 939702 | 933502 | 1041880 |
| | 100% | 89.58% | 79.82% | 67.07% | 52% | 36.6% | 31.35% | 21.78% | 13.42% | 9.62% | 10.22% | 0% |
| B | 7616 | 115847 | 229409 | 367055 | 561516 | 661504 | 737068 | 853233 | 885259 | 935415 | 968576 | 1071667 |
| | 100% | 89.36% | 78.36% | 65.04% | 46.22% | 36.54% | 29.23% | 17.99% | 14.88% | 10.03% | 6.82% | 0% |
| C | 5003 | 125213 | 229360 | 382045 | 529553 | 674042 | 749780 | 874120 | 942952 | 959358 | 976911 | 1074307 |
| | 100% | 88.45% | 78.36% | 63.59% | 49.3% | 35.33% | 28% | 15.96% | 9.3% | 7.72% | 6.02% | 0% |
| D | 4755 | 123605 | 232610 | 460226 | 559590 | 682861 | 773384 | 919377 | 905782 | 928034 | 1033152 | 1086941 |
| | 100% | 88.6% | 78.05% | 56.02% | 46.4% | 34.48% | 25.72% | 11.6% | 12.9% | 10.75% | 0.575% | 0% |

Control 1: without enzyme
Control 2: without inhibitor

TABLE 5k
ACE Inhibitor Assay
Peptid S548 (PLPEVPP)

| | Control 1 | | | | | | | | | | | Control 2 |
|---|---|---|---|---|---|---|---|---|---|---|---|---|
| | 200 μM | | | | | | 6.25 | 3.125 | 1.5625 | 0.78125 | 0.39 | (no |
| | Inhibitor | 200 μM | 100 μM | 50 μM | 25 μM | 12.5 μM | μM | μM | μM | μM | μM | Inhibitor) |
| A | 5359 | 101322 | 168452 | 278402 | 402465 | 572641 | 628007 | 719509 | 773857 | 787559 | 812986 | 760427 |
| | 100% | 89,28% | 81,8% | 69,55% | 55,73% | 36,76% | 30,6% | 20,4% | 14,35% | 12,82% | 9,98% | 0% |
| B | 6630 | 108115 | 181416 | 291507 | 411233 | 668264 | 644083 | 735840 | 672595 | 736956 | 850082 | 813278 |
| | 100% | 88,52% | 80,36% | 68,1% | 54,75% | 37,25% | 28,8% | 18,58% | 25,63% | 18,46% | 5,85% | 0% |
| C | 4358 | 108968 | 194177 | 326054 | 394114 | 582524 | 676136 | 756404 | 802519 | 848448 | 861072 | 789532 |
| | 100% | 88,43% | 78,94% | 64,24% | 56,66% | 35,66% | 25,23% | 16,29% | 11,15% | 6,03% | 4,63% | 0% |
| D | 4280 | 113955 | 204171 | 343249 | 413347 | 619407 | 699343 | 788637 | 857919 | 878121 | 990310 | 812061 |
| | 100% | 87,87% | 77,82% | 62,33% | 54,52% | 31,55% | 22,65% | 12,7% | 4,98% | 2,73% | −9,77% | 0% |

Control 1: without enzyme
Control 2: without inhibitor

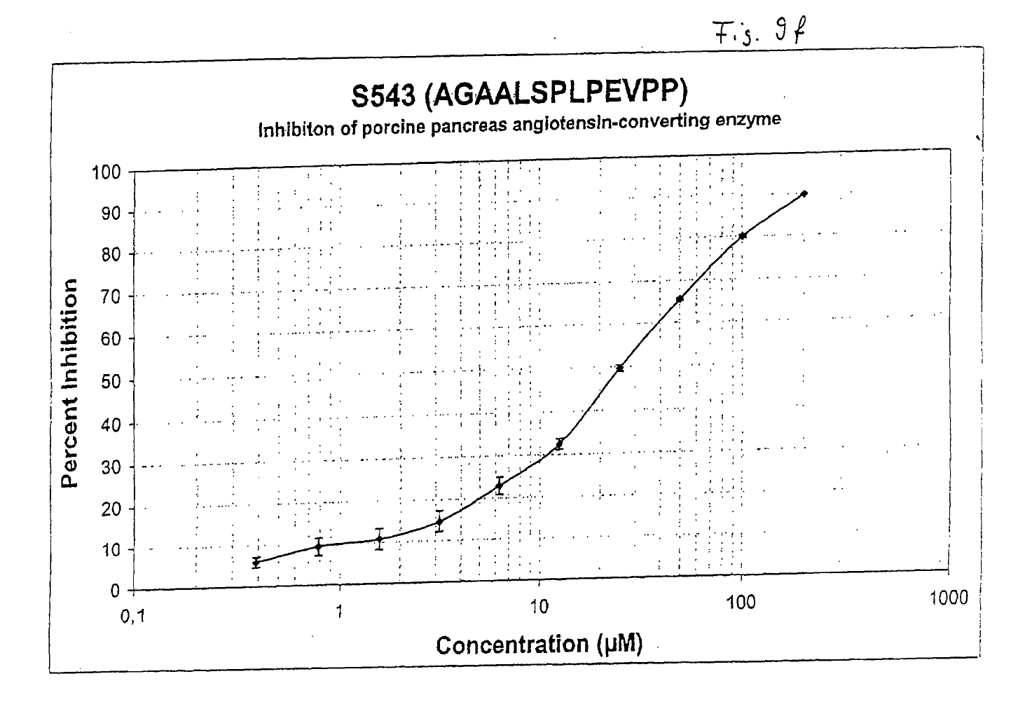

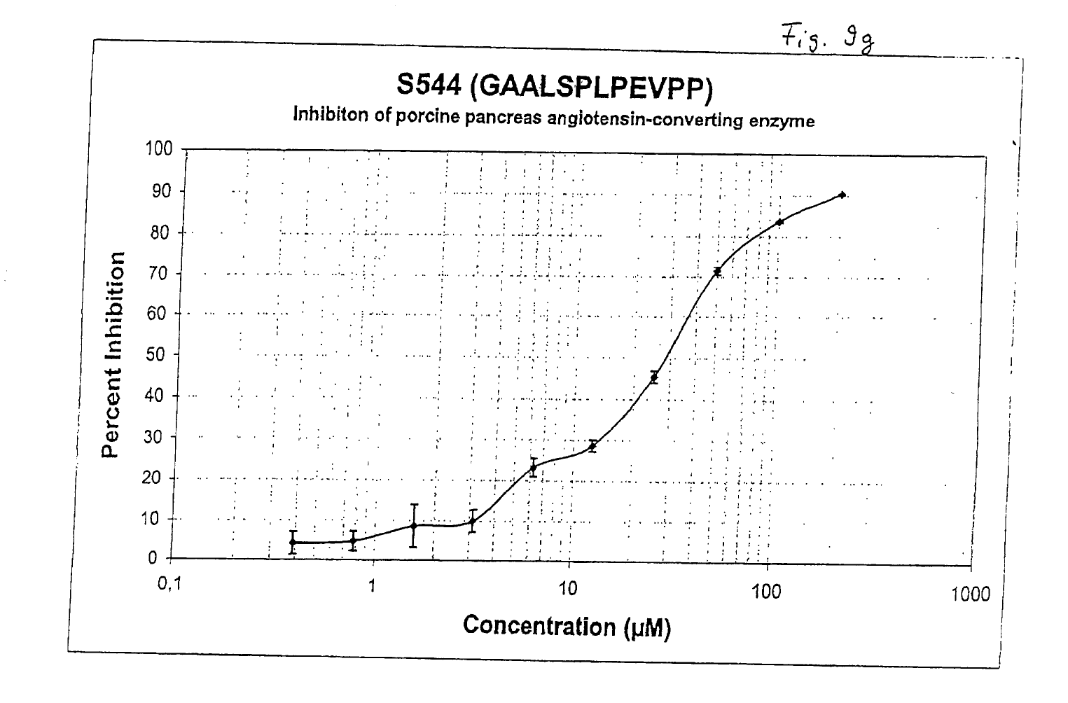

The decapeptide S545 (which is a synonym to S605) showing the highest activity was used in further experiments in order to prove the bradykinin-potentiating effect. These experiments were conducted with normotensive rats and the short-time decrease of blood pressure induced by bradykinin (and caused by vasodilatation) was measured. These experiments indicate a concentration dependent bradykinin-potentiating of this peptide. Furthermore, S545 (also corresponds to proANP₄₈₋₅₇) was also tested in vivo at hypertensive rats (FIG. 7).

In the in vivo experiments with normotensive rats the animals were previously anaesthetized and after blood pressure stabilization treated with a constant amount of bradykinin. Subsequently, either Captopril or human BPP were administered, again followed by a constant amount of bradykinin. Afterwards the maximum blood pressure drop was determined and, on the basis of bradykinin as a factor 1, the potentiating factor was determined. The results are presented in tables 3a and 3b. In the same way, the systolic and diastolic blood pressure, the percentage middle pressure decrease and the time required to come back to the baseline values were measured (see tables 4a to 4j). A detailed experimental description is given below.

Bradykinin potentiating effect of Captopril

Enhancement of the blood pressure reducing effects of Bradykinin

TABLE 3a

| | maximal drop of | |
|---|---|---|
| combination tested^{1,2)} | blood pressure | potentiating factor³⁾ |
| Bradykinin 2.5 × 10⁻⁶ M | 28% ± 4.5 | 1 |
| (n = 3) | | |
| Captopril 1.5 × 10⁻⁴ M + | 56% ± 17.3 | 2 |
| Bradykinin 2.5 × 10⁻⁶ M | | |
| (n = 3) | | |

¹⁾M (Bradykinin) = 1060.2 g/mol; M (Captopril) = 217.3 g/mol
²⁾the corresponding substance doses refer to 1 kg rat
³⁾factor, by which the blood pressure reducing effect of Bradykinin is potentiated (Bradykinin = 1)

Bradykinin potentiating effect of S605

Enhancement of the blood pressure reducing effects of Bradykinin

TABLE 3b

| | maximal drop of | |
|---|---|---|
| combination tested^{1,2)} | blood pressure | potentiating factor³⁾ |
| Bradykinin 2.5 × 10⁻⁶ M | 31.3% ± 5.9 | 1 |
| (n = 9) | | |
| S605 1.5 × 10⁻⁴ M + | 38.9% ± 8.3 | 1.24 |
| Bradykinin 2.5 × 10⁻⁶ M | | |
| (n = 3) | | |
| S605 1 × 10⁻³ M + | 48.5% | 1.49 |
| Bradykinin 2.5 × 10⁻⁶ M | | |
| (n = 1) | | |
| S605 1.5 × 10⁻³ M + | 50.5% ± 5.2 | 1.61 |
| Bradykinin 2.5 × 10⁻⁶ M | | |
| (n = 3) | | |
| S605 5 × 10⁻³ M + | 63.8% ± 3.6 | 2.04 |
| Bradykinin 2.5 × 10⁻⁶ M | | |
| (n = 3) | | |

¹⁾M (Bradykinin) = 1060.2 g/mol; M (S605) = 1019.2 g/mol; Sequenz: Ala-Leu-Ser-Pro-Leu-Pro-Glu-Val-Pro-Pro
²⁾the corresponding substance doses refer to 1 kg rat
³⁾factor, by which the blood pressure reducing effect of Bradykinin is potentiated (Bradykinin = 1)

TABLE 4a

| | | |
|---|---|---|
| S605 1.5 × 10⁻⁴ M | Rat (female) 238 g | 14/09/1999 |

| Blood pressure normal (mm Hg) | |
|---|---|
| systolic | 82 |
| diastolic | 62 |
| P_{M} (middle pressure) = | 70 |
| (Pₛ − P_{d}) × 0.42 + P_{d} | |

| Bradykinin 2.5 × 10⁻⁶ M | Blood pressure minimal (mm Hg) |
|---|---|
| systolic | 64 |
| diastolic | 42 |
| P_{M} (middle pressure) = | 51 |
| (Pₛ − P_{d}) × 0.42 + P_{d} | |
| ΔP (middle pressure drop) | 19 (27.1%) |
| time to reach the baseline value (t = 19 s) | |
| S605 1.5 × 10⁻⁴ M + | |
| Bradykinin 2.5 × 10⁻⁶ M | Blood pressure minimal (mg Hg) |
| systolic | 60 |
| diastolic | 40 |
| P_{M} (middle pressure) = | 48 |
| (Pₛ − P_{d}) × 0.42 + P_{d} | |
| ΔP (middle pressure drop) | 22 (31.4%) |

time to reach the baseline value (t = 26 s)

TABLE 4b

| | | |
|---|---|---|
| S605 1.5 × 10⁻⁴ M | Rat (female) 214 g | 13/07/1999 |

| Blood pressure normal (mm Hg) | |
|---|---|
| systolic | 79 |
| diastolic | 57 |
| P_{M} (middle pressure) = | 66 |
| (Pₛ − P_{d}) × 0.42 + P_{d} | |

| Bradykinin 2.5 × 10⁻⁶ M | Blood pressure minimal (mm Hg) |
|---|---|
| systolic | 51 |
| diastolic | 30 |
| P_{M} (middle pressure) = | 39 |
| (Pₛ − P_{d}) × 0.42 + P_{d} | |
| ΔP (middle pressure drop) | 27 (41.2%) |
| time to reach the baseline value (t = 26 s) | |
| S605 1.5 × 10⁻⁴ M + | |
| Bradykinin 2.5 × 10⁻⁶ M | Blood pressure minimal (mg Hg) |
| systolic | 46 |
| diastolic | 26 |
| P_{M} (middle pressure) = | 34 |
| (Pₛ − P_{d}) × 0.42 + P_{d} | |
| ΔP (middle pressure drop) | 32 (47.9%) |

time to reach the baseline value (t = 36 s)

TABLE 4c

| | | |
|---|---|---|
| S605 1.5 × 10⁻⁴ M | Rat (female) 238 g | 08/07/1999 |

| Blood pressure normal (mm Hg) | |
|---|---|
| systolic | 65 |
| diastolic | 44 |
| P_{M} (middle pressure) = | 53 |
| (Pₛ − P_{d}) × 0.42 + P_{d} | |

| Bradykinin 2.5 × 10⁻⁶ M | Blood pressure minimal (mm Hg) |
|---|---|
| systolic | 44 |
| diastolic | 32 |
| P_{M} (middle pressure) = | 37 |
| (Pₛ − P_{d}) × 0.42 + P_{d} | |
| ΔP (middle pressure drop) | 16 (29.8%) |
| time to reach the baseline value (t = 11 s) | |
| S605 1.5 × 10⁻⁴ M + | |
| Bradykinin 2.5 × 10⁻⁶ M | Blood pressure minimal (mg Hg) |
| systolic | 40 |
| diastolic | 28 |
| P_{M} (middle pressure) = | 33 |
| (Pₛ − P_{d}) × 0.42 + P_{d} | |
| ΔP (middle pressure drop) | 20 (37.4%) |

time to reach the baseline value (t = 23 s)

TABLE 4d

| | | |
|---|---|---|
| S605 1.5 × 10⁻³ M | Rat (female) 230 g | 15/07/1999 |

| Blood pressure normal (mm Hg) | |
|---|---|
| systolic | 83 |
| diastolic | 64 |
| P_{M} (middle pressure) = | 72 |
| (Pₛ − P_{d}) × 0.42 + P_{d} | |

| Bradykinin 2.5 × 10⁻⁶ M | Blood pressure minimal (mm Hg) |
|---|---|
| systolic | 62 |
| diastolic | 44 |
| P_{M} (middle pressure) = | 52 |
| (Pₛ − P_{d}) × 0.42 + P_{d} | |
| ΔP (middle pressure drop) | 20 (28.4%) |
| time to reach the baseline value (t = 34 s) | |
| S605 1.5 × 10⁻³ M + | |
| Bradykinin 2.5 × 10⁻⁶ M | Blood pressure minimal (mg Hg) |
| systolic | 49 |
| diastolic | 34 |
| P_{M} (middle pressure) = | 40 |
| (Pₛ − P_{d}) × 0.42 + P_{d} | |
| ΔP (middle pressure drop) | 32 (44.6%) |

time to reach the baseline value (t = 46 s)

TABLE 4e

| | | |
|---|---|---|
| S605 1.5 × 10⁻³ M | Rat (female) 230 g | 14/07/1999 |

| Blood pressure normal (mm Hg) | |
|---|---|
| systolic | 90 |
| diastolic | 64 |
| P_{M} (middle pressure) = | 75 |
| (Pₛ − P_{d}) × 0.42 + P_{d} | |

| Bradykinin 2.5 × 10⁻⁶ M | Blood pressure minimal (mm Hg) |
|---|---|
| systolic | 66 |
| diastolic | 41 |
| P_{M} (middle pressure) = | 52 |
| (Pₛ − P_{d}) × 0.42 + P_{d} | |
| ΔP (middle pressure drop) | 23 (30.9%) |
| time to reach the baseline value (t = 22 s) | |
| S605 1.5 × 10⁻³ M + | |
| Bradykinin 2.5 × 10⁻⁶ M | Blood pressure minimal (mg Hg) |
| systolic | 45 |
| diastolic | 26 |
| P_{M} (middle pressure) = | 34 |
| (Pₛ − P_{d}) × 0.42 + P_{d} | |
| ΔP (middle pressure drop) | 41 (54.4%) |

time to reach the baseline value (t = 57 s)

TABLE 4f

| S605 1 × 10⁻³ M | | |
|---|---|---|
| and 5 × 10⁻³ M | Rat (female) 230 g | 09/07/1999 |

Blood pressure normal (mm Hg)

| systolic | 91 |
|---|---|
| diastolic | 66 |
| P_{M} (middle pressure) = | 76 |
| (Pₛ − P_{d}) × 0.42 + P_{d} | |
| Bradykinin 2.5 × 10⁻⁶ M | Blood pressure minimal (mm Hg) |
| systolic | 67 |
| diastolic | 32 |
| P_{M} (middle pressure) = | 47 |
| (Pₛ − P_{d}) × 0.42 + P_{d} | |
| ΔP (middle pressure drop) | 30 (39%) |
| time to reach the baseline value (t = 18 s) | |
| S605 1 × 10⁻³ M + | |
| Bradykinin 2.5 × 10⁻⁶ M | Blood pressure minimal (mg Hg) |
| systolic | 55 |
| diastolic | 28 |
| P_{M} (middle pressure) = | 39 |
| (Pₛ − P_{d}) × 0.42 + P_{d} | |
| ΔP (middle pressure drop) | 37 (48.5%) |

time to reach the baseline value (t = 31 s)

TABLE 4g

| S605 5 × 10⁻³ M + | |
|---|---|
| Bradykinin 2.5 × 10⁻⁶ M | Blood pressure minimal (mg Hg) |
| systolic | 36 |
| diastolic | 16 |
| P_{M} (middle pressure) = | 24 |
| (Pₛ − P_{d}) × 0.42 + P_{d} | |
| ΔP (middle pressure drop) | 52 (68%) |

time to reach the baseline value (t = 36 s)
following short overreaction at maximum of 112/84

TABLE 4h

| | | |
|---|---|---|
| S605 5 × 10⁻³ M | Rat (female) 218 g | 12/07/1999 |

| Blood pressure normal (mm Hg) | |
|---|---|
| systolic | 119 |
| diastolic | 98 |
| P_{M} (middle pressure) = | 107 |
| (Pₛ − P_{d}) × 0.42 + P_{d} | |

| | |
|---|---|
| Bradykinin 2.5 × 10⁻⁶ M | Blood pressure minimal (mm Hg) |

| | |
|---|---|
| systolic | 92 |
| diastolic | 70 |
| P_{M} (middle pressure) = | 79 |
| (Pₛ − P_{d}) × 0.42 + P_{d} | |
| ΔP (middle pressure drop) | 27 (25.6%) |

| time to reach the baseline value (t = 36 s) | |
|---|---|
| S605 5 × 10⁻³ M + | |
| Bradykinin 2.5 × 10⁻⁶ M | Blood pressure minimal (mg Hg) |
| systolic | 52 |
| diastolic | 32 |
| P_{M} (middle pressure) = | 40 |
| (Pₛ − P_{d}) × 0.42 + P_{d} | |
| ΔP (middle pressure drop) | 66 (62.1%) |

time to reach the baseline value (t = 87 s)
following short overreaction at maximum of 154/112

TABLE 4i

| | | |
|---|---|---|
| S605 5 × 10⁻³ M | Rat (female) 211 g | 13/09/1999 |

| Blood pressure normal (mm Hg) | |
|---|---|
| systolic | 128 |
| diastolic | 104 |
| P_{M} (middle pressure) = | 114 |
| (Pₛ − P_{d}) × 0.42 + P_{d} | |

| Bradykinin 2.5 × 10⁻⁶ M | Blood pressure minimal (mm Hg) |
|---|---|
| systolic | 98 |
| diastolic | 78 |
| P_{M} (middle pressure) = | 86 |
| (Pₛ − P_{d}) × 0.42 + P_{d} | |
| ΔP (middle pressure drop) | 28 (24.5%) |
| time to reach the baseline value (t = 16 s) | |
| S605 5 × 10⁻³ M + | |
| Bradykinin 2.5 × 10⁻⁶ M | Blood pressure minimal (mg Hg) |
| systolic | 56 |
| diastolic | 35 |
| P_{M} (middle pressure) = | 44 |
| (Pₛ − P_{d}) × 0.42 + P_{d} | |
| ΔP (middle pressure drop) | 70 (61.4%) |

time to reach the baseline value (t = 44 s)
following short overreaction at maximum of 141/108

TABLE 4j

| | | |
|---|---|---|
| S605 1.5 × 10⁻³ M | Rat (female) 216 g | 13/09/1999 |

| Blood pressure normal (mm Hg) | |
|---|---|
| systolic | 98 |
| diastolic | 71 |
| P_{M} (middle pressure) = | 82 |
| (Pₛ − P_{d}) × 0.42 + P_{d} | |

| Bradykinin 2.5 × 10⁻⁶ M | Blood pressure minimal (mm Hg) |
|---|---|
| systolic | 68 |
| diastolic | 42 |
| P_{M} (middle pressure) = | 53 |
| (Pₛ − P_{d}) × 0.42 + P_{d} | |
| ΔP (middle pressure drop) | 29 (35.4%) |
| time to reach the baseline value (t = 13 s) | |
| S605 1.5 × 10⁻³ M + | |
| Bradykinin 2.5 × 10⁻⁶ M | Blood pressure minimal (mg Hg) |
| systolic | 55 |
| diastolic | 28 |
| P_{M} (middle pressure) = | 39 |
| (Pₛ − P_{d}) × 0.42 + P_{d} | |
| ΔP (middle pressure drop) | 43 (52.4%) |

time to reach the baseline value (t = 20 s)
following short overreaction at maximum of 106/80

Interestingly, the bradykinin-potentiating effect in vivo requires only a 10-fold higher molecular concentration of hBPP in comparison to Captopril, whereas in the ACE-assay a dose 1000-fold higher is necessary to reach the same effect as with Captopril. This leads to the conclusion, that besides the ACE-inhibition a second activity for bradykinin-potentiation must exist in vivo.

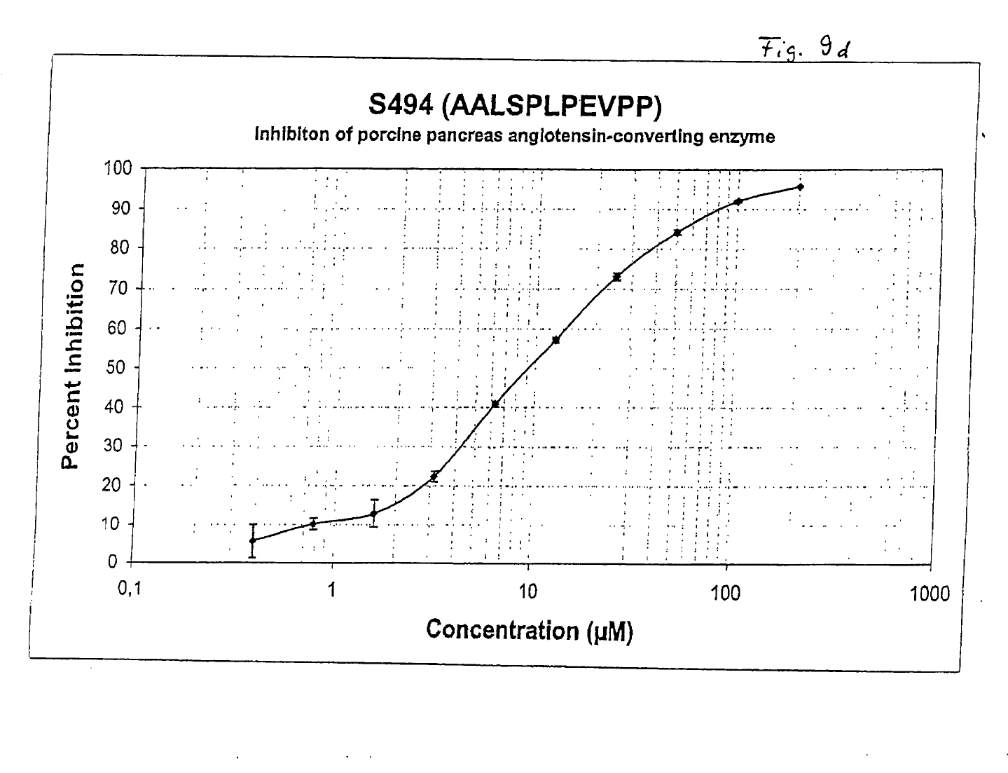

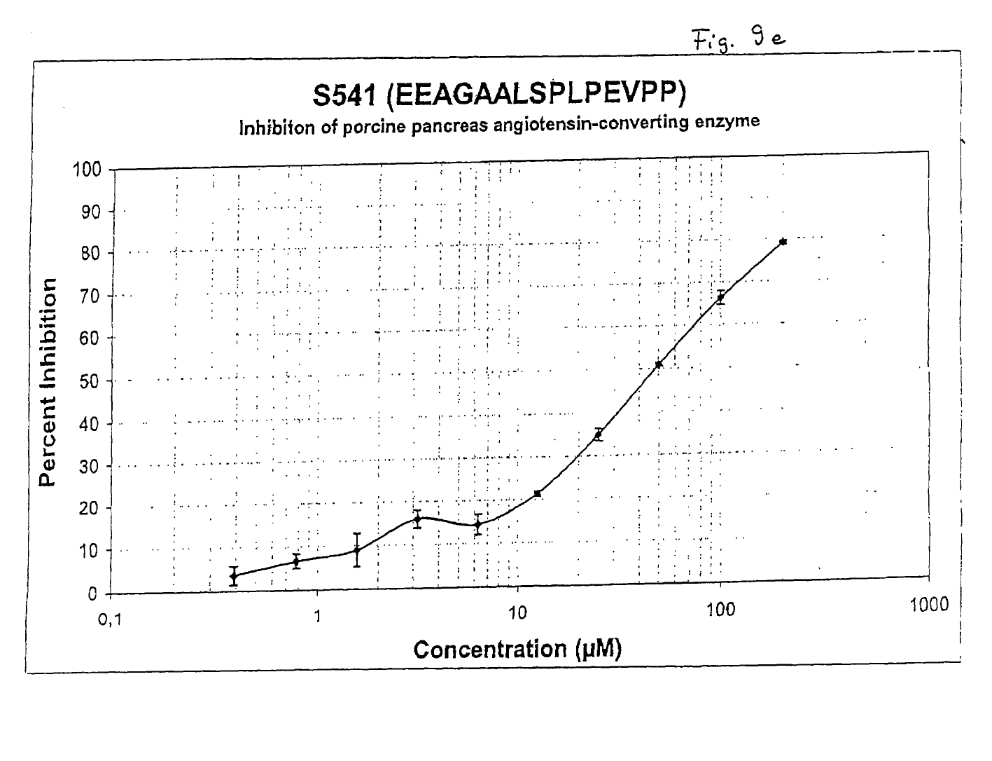

A possible explanation can be given by BPPs interacting with a membrane bound receptor, e.g. thus acting as allosteric regulators at the bradykinin B2-receptor (FIG. 6). Thus, findings derived from studies conducted with the peptides according to the invention might serve for the validation of novel drug targets—namely the membrane bound receptor—and therefore as well might serve for the development of novel therapeutics for treatment of hypertension.

Experimental Procedure:

1. ACE-Inhibition-Assay

The principle of the ACE-inhibition-assay was established by Chejung, H. S., Cushman, D. W.: “Inhibition of homogenous angiotensin converting enzyme of rabbit lung by synthetic venom peptides of Bothrops jararaca.” in Biochimica et Biophysica Acta 293, 451-563 (1973).

The peptides used in the assay were synthesized and purchased from Biosynthan (Berlin) and showed a purity of more than 92%. Captopril and the known snake-BPP BPP9a were purchased from Sigma.

The assay was performed in 96 well HTRF-plates (Packard). The concentrations used for each well were:

1 mU ACE from pig kidney (EC 3.4.15.1 (Sigma)) in 30 μl of assay buffer (25 mM HEPES; 0,3 M NaCl, pH 8,2) and 20 μl inhibitor dissolved in assay buffer (0,35-200 μM). The reaction was started by adding 50 μl of 2 mM hippuryl-histidyl-leucin (Sigma) in assay buffer. The reaction was performed at room temperature (22° C.) for 30 minutes and terminated by adding 50 μl of 2 M NaOH. Afterwards 50 μl of 0,5% ortho-phtalaldehyd (Sigma) in methanol were added, 5 minutes later followed by 50 μl of 2,5 M HCl-solution to stabilize the developed fluorescence product. Fluorescence was determined within the following 15 minutes using a 1420 Victor Multilabelcounter (Wallac) with an activating wavelength of λ=3557 30 nm and an emission wavelength of λ=515 nm.

The results were used to calculate the % inhibition. The IC₅₀-values were determined graphically from a curve showing the dose-reaction ratio.

In order to minimize errors due to time-dependent differences, all pipetting steps were performed in a precise time schedule.

2. Measurement of Blood Pressure in Normotensive and Hypertensive Rats

The trials were conducted on female anaesthetized normotensive Wister rats with weight ranging from 210 to 240 g.

The peptide pANP₄₈₋₅₇ used for the in vivo assays was synthesized by Biosynthan (Berlin) with a HPLC-determined purity of 98%.

Anaesthetizing was performed by i.p.-administration of 1 ml/kg Ketavit (100 mg/ml)+Rampun (2%).

In order to conduct the blood pressure measurement, a polyethylene catheter was introduced into the Aorta femuralis and connected with a pressure transducer. A Statham-P23A pressure-transducer was linked to a Gould Polygraph and used to determine the arterial blood pressure. A Hg-manometer was used to calibrate the system.

All substances were dissolved and diluted in a physiological sodium chloride solution and introduced into the Vena jugularis via a catheter.

After stabilization of blood pressure (after about 10 minutes) 1 mg/kg body weight of a 2,5 μM bradykinin-solution (Sigma) was administered. After an interval of 5 minutes a dose of 1 mg/kg Captopril (Sigma) or pANP₄₈₋₅₇, immediately followed by 1 mg/kg of a 2,5 μM bradykinin-solution was administered.

The middle pressure was determined from the measured diastolic (p_{d}) and systolic (pₛ) blood pressure by using the following formula: pₘ=(pₛ−P_{d})×0,42+p_{d}.

The experimental procedure using the hypertensive rats corresponded to the experiments using normotensive Wistar-rats (see FIG. 6). The experimental animals were Wistar-Kyoto rats. These rats were rendered hypertensive by narrowing the left kidney artery. This classic high pressure model (two-kidney-one-cliprenovascular hypertension) is independent from the rat stem used.

II. Exendines

In the past 20 years, a number of peptides (Helodermin, Helospectine, exendin 3 and exendin 4) were discovered in the venom of the lizard family Helodermatidae (comprising the species Heloderma suspectum and Heloderma horridum) which interact with cell membrane receptors in mammalian tissues. Exendin-3 (Ser2-Asp3) differs from exendin-4 (Gly2-Glu3) in respect of two amino acids. This structural difference also causes a difference in activity. Exendin-3 interacts with VIP-receptors and GLP-1 receptors, exendin-4 only reacts with GLP-1 receptors.

These peptides with biochemical effects similar to glucagon induce specific physiological reactions, e.g. an increased insulin secretion and a stimulation of the pancreas' island cells by exendin-4 in diabetic rodents.

Investigating the activity of these peptides revealed novel receptors and receptor types and broadened the understanding of mammalian physiology. There is a remarkable similarity between lizard peptides with peptides of the glucagon/vasoactive intestinal peptide (VIP)/secretin superfamily.

In the course of high throughput sequencing of a cDNA-library derived from the salivary gland of Heloderma horridum the following cDNA-sequences identified:

>helo_all.0.1085 (exendin-1)

| | |
|---|---|
| 1 | cttcagacgt cactgctgaa acctctgctc tgagtttggt |

gtctgtgcag

| | |
|---|---|
| 51 | aagaggagat gaaaagcatc ctttggctgt gtgtttttgg |

gctgctcatt

| | |
|---|---|
| 101 | gcaactttat tccctgtcag ctggcaaatg gctatcaaat |

ccaggttatc

| | |
|---|---|
| 151 | ttctgaagac tcagaaacag accaaagatt gcttgagagt |

aagcgacatt

| | |
|---|---|
| 201 | ctgatgcaac atttactgcg gagtattcga agcttctagc |

aaagttggca

| | |
|---|---|
| 251 | ctacagaagt atcttgagag cattcttgga tccagtacat |

caccacgtcc

| | |
|---|---|
| 301 | gccatcgcgt taaggtcttt gagttgtgga acacgacaca |

catctgatgt

| | |
|---|---|
| 351 | ttgacgacca ttttgaagaa aagtttcggg caatatgtta |

catgtctttg

| | |
|---|---|
| 401 | tttccaatta gtgagctaca aaggctttct caattaaaaa |

aaaattgaag

| | |
|---|---|
| 451 | tcatgcaa |

>helo_all.0.564 (exendin-3)

| | |
|---|---|
| 1 | ctggctggtc ttcagaagtc actgctcaaa tctctattct |

gaatttggtg

| | |
|---|---|
| 51 | cctgtgcaaa ggagaagatg aaaatcatcc tgtggctgtg |

tgttttcggg

| | |
|---|---|
| 101 | ctgttccttg caactttatt ccctgtcagc tggcaaatgc |

ctgttgaatc

| | |
|---|---|
| 151 | tgggttgtct tctgaggatt ctgcaagctc agaaagcttt |

gcttcgaaga

| | |
|---|---|
| 201 | ttaagcgaca tagtgatgga acatttacca gtgacttgtc |

aaaacagatg

| | |
|---|---|
| 251 | gaagaggagg cagtgcggtt atttattgag tggcttaaga |

acggaggacc

| | |
|---|---|
| 301 | aagtagcggg gcacctccgc catcgggtta aggtctttca |

attgtggaac

| | |
|---|---|
| 351 | aagacacaca cctgatgttt gatgaccatt ttaaagaaat |

gtttccagca

| | |
|---|---|
| 401 | atacgtcaca tgtctttgtt tccaattagt gagcgacaca |

gcctttctta

| | |
|---|---|
| 451 | attaaaaaat tgaagtcatg c |

A comparison of these sequences with known sequences present in public data bases allowed for the following result:

1. for helo_all.0.1085 (exendin-1)

BLASTX 2.1.3 [Apr-1-2001]

Reference: Altschul, Stephen F., Thomas L. Madden, Alejandro A. Schaffer, Jinghui Zhang, Zheng Zhang, Webb Miller, and David J. Lipman (1997),

“Gapped BLAST and PSI-BLAST: a new generation of protein database search

programs”, Nucleic Acids Res. 25:3389-3402.

Query=/homes/ts/heloderma/exendine/HELO1085.SEQ

(458 letters)

Database: ncbi_nr

1,632,343 sequences; 523,647,861 total letters

Score E

Sequences producing significant alignments: (bits) Value . . .

NR:GI-1916067 Begin: 1 End: 71

!(U77613) exendin 4 [Heloderma suspectum] 74 3e-12

NR:GI-2851623 Begin: 1 End: 71

!EXENDIN-4 PRECURSOR 74 3e-12

NR:GI-69269 Begin: 1 End: 28

!exendin-1-Mexican beaded lizard 42 0.014

NR:GI-119675 Begin: 1 End: 28

!EXENDIN-1 (HELOSPECTINS I AND II) 42 0.014

NR:GI-556438 Begin: 115 End: 155

!(L36641) vasoactive intestinal peptide [Meleagris g . . . 38 0.21

NR:GI-487633 Begin: 115 End: 155

!(U09350) vasoactive intestinal peptide [Gallus gallus] 38 0.21

NR:GI-1353216 Begin: 115 End: 155

!VASOACTIVE INTESTINAL PEPTIDE PRECURSOR (VIP) 38 0.21

NR:GI-1174967 Begin: 115 End: 155

!VASOACTIVE INTESTINAL PEPTIDE PRECURSOR (VIP) 38 0.21

NR:GI-14549660 Begin: 111 End: 158

!(AF321243) growth hormone-releasing hormone/pitui . . . 36 0.60

NR:GI-1352710 Begin: 110 End: 157

!GLUCAGON-FAMILY NEUROPEPTIDES PRECURSOR [CONTAINS: . . . 36 0.60

2. for helo_all.0.564 (exendin-3)

Reference: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search

programs”, Nucleic Acids Res. 25:3389-3402.

Query=/homes/ts/heloderma/exendine/HEL0564.SEQ

(471 letters)

Database: ncbi_nr

1,632,343 sequences; 523,647,861 total letters

Score E

Sequences producing significant alignments: (bits) Value . . .

NR:GI-1916067 Begin: 1 End: 75

!(U77613) exendin 4 [Heloderma suspectum] 116 4e-25

NR:GI-2851623 Begin: 1 End: 75

!EXENDIN-4 PRECURSOR 116 4e-25

NR:GI-279624 Begin: I End: 28

!exendin-3-Mexican beaded lizard 61 2e-08

NR:GI-119677 Begin: 1 End: 28

!EXENDIN-3 61 2e-08

NR:GI-17942697 Begin: 1 End: 28

!Chain A, Solution Structure Of Exendin-4 In 30-Vo . . . 58 2e-07

NR:GI-279625 Begin: 1 End: 28

!exendin-4-Gila monster 58 2e-07

NR:GI-248418 Begin: 1 End: 28

!exendin-4 [Heloderma suspectum, venom, Peptide, 39 aa] 58 2e-07

NR:GI-121471 Begin: 9 End: 79

!GLUCAGON II PRECURSOR [CONTAINS: GLICENTIN-RELATED . . . 45 0.001

NR:GI-121471 Begin: 87 End: 115

!GLUCAGON II PRECURSOR [CONTAINS: GLICENTIN-RELATED . . .

NR:GI-279617 Begin: 9 End: 79

The results of the sequence comparisons indicate, that helo_all.0.1085 encodes for the so far unknown precursor protein of exendin-1 (=Helospectin) and helo_all.0.564 encodes for the so far unknown precursor protein of exendin-3 in Heloderma horridum.

There is a further similarity of these sequences to VIP and glucagon, which is evident from the following figure:

| | |
|---|---|
| human | HSQGTFTSDYSKYLDSRRAQDFVQWLMNT |
| Glucagon | |
| human GLP-1 | HAEGTFTSDVSSYLEGQAADEFIAWLVKGR |
| exendin-3 | |
| | HSDGTFTSDLSKQMEEEAVRLFIEWLKNGGPSSGA |
| PPPS | |
| human GLP-2 | HADGSFSDEMNTILDNLAARDFINWLIQTKITD |
| Consensus | Ha#GtFts#.s..$#..aardF!.WL..t. |
| human VIP | HSDAVFTDNYTRLRKQMAVKKYLNSILN |
| exendin-1 | |
| | HSDATFTAEYSKLLAKLALQKYLESILGSSTSPRPPS |
| S | |
| Consensus | HSDAtFTa#YsrLraq$AlqKYL#SILn........ |

It is said that exendin-1 was originally isolated from the venom of H. suspectum. However, these results indicate, that exendin-1 is produced by H. horridum. The probes of venom were purchased from Sigma. Probably the probes were exchanged or even intermingled resulting in this wrong classification.

With a search using the two cDNA-sequences of H. horridum in a human cDNA-library cDNA-clones encoding for the precursor-proteins of the above mentioned human peptides could be found.

1. Method for identifying a substance being pharmacologically active in a target organism, comprising the following steps:
defining a preferred physiological property of the desired substance
searching for and selecting of a reference organism or -tissue or parts thereof comprising the desired property or at least properties being essentially functional similar thereto
identifying a biologically active substance or parts thereof underlying the desired property of the reference organism or tissue or parts thereof and possibly identifying the genetic information or parts thereof encoding for said biological substance,
identifying orthologous structures or substances and possibly genetic information within the target organism.

2. Method according to claim 1, wherein a EST-library of the selected reference organism or -tissue or parts thereof is established and the biologically active substance in the reference organism is identified.

3. Method according to claim 2, characterized in a comparative structure- and sequence analysis between the EST-library of the reference organism and sequence information of the target organism.

4. Method according to claim 3, characterized in the identification of the orthologous substance in the target organism.

5. Method according to claim 1 or 2, characterized in employing at least two reference organisms.

6. Method according to claim 5, characterized in a comparative structure- and sequence analysis between the reference organism and determination of a conserved structure underlying the biologically active substance of the reference organism.

7. Method according to claim 6, characterized in comparing the conserved structure with sequence information in the target organism and identification of a structure in the target organism, which is essentially orthologous to the conserved structure.

8. Method according to claim 4 or 7, characterized in a modification of the orthologous substance of the target organism.

9. Method according to claim 8, characterized in using structure-based design for optimizing the desired biological properties of the orthologous substance.

10. Polypeptide manufactured by a method according to one of the claims 1 to 9.

11. Oligonucleotide encoding for a polypeptide according to claim 10.

12. Pharmaceutical composition comprising a polypeptide according to claim 10.

13. Use of the polypeptide according to claim 10 for the preparation of a pharmaceutical drug.

14. Use of the method according to one of the claims 1 to 7 or of a polypeptide according to claim 10 or of an oligonucleotide according to claim 11 for identifying and validating a drug target.

15. Use of a method according to one of the claims 1 to 9 or of a polypeptide according to claim 10 or of an oligonucleotide according to claim 11 for identifying a lead structure of a pharmacologically active substance.

16. Method for providing a validated drug target characterized in the use of a polypeptide according to claim 10 or a polynucleotide according to claim 11.

17. Drug target provided by the method according to claim 16.

18. Method for providing a pharmacologically active substance comprising the following steps:
preparing a polypeptide according to one of the claims 1 to 9
validating the polypeptide as a drug target
developing a biologically active ligand of the drug target

19. Pharmacologically active substance manufactured by a method according to claim 18.

20. Pharmacological composition comprising a substance according to claim 19.

21. Use of a substance according to claim 19 for manufacturing a drug.

22. Polypeptide or derivative thereof according to SEQ. ID. No 2.

23. Oligonucleotide or derivative thereof according to SEQ. ID. No 1.

24. Peptide according to one of the sequences SEQ. ID. No 3 to 15.

25. Use of a peptide according to claim 22 or 24 or of an oligonucleotide according to claim 23 in a method according to claim 16 or 18.
